SEQUENCE LISTING (14 sequences)

SEQ ID NO: 1 - DNA, 2445 bp, Cannabis sativa
gtcaataaaaattagtattatattattacaattccaaataataataataaagaaaaggtttgtgattgtggtggttcgtttaaagccatcttcatcaaatatttgccacacaccactgaacctcattctgtttctcaaccatggcggccgccaccactaatgctccaacctcctccttcctcacaaagacctctactttctcatcctcctccacctcctcttcaaaacccattctcagattcatactcccattctcctcatctccccataaccccgctctcttccgccgcccactccatctcaccaacacctccctccctcgccgaaaccctccttctgccgtaacaactccaacccccaccaccatttcctccgaccccgacaccttcgtctcccgattcggtcccgacgagccacgaaaaggcgcagatgttctcgtcgaagccctcgagcgcgaaggtgttaaagacgtcttcgcttacccaggtggtgcttccatggagatccaccaagctctgaccaggtctagcagcattcgaaatgtcctccctcgacacgagcagggtggtatctttgcggctgagggttacgctcgctcctctggtcttccaggtgtttgtattgcgacctccggccccggcgccaccaacttggtcagtggcttagccgacgcgctccttgatagtgttcctttagtcgccattactggacaagtcccaaggaggatgatcggtactgatgcgtttcaggaaacccctattgttgaggtaacaagatctattacaaagcataattaccttgttcttgatgtggaagacattcctagggtagttaaagaagctttctttttagccaaatcgggtcgacctggtcctgttttgattgatgtacctaaagatatacaacaacagcttgttgttcccaattgggatcaacccatgaagctacctgggtacttagctaggctccccaagctccctaatgaagctcatttggagcagattgtgaggctgatttctgagtcaaagaagccagttttgtatgttggtggtgggtgtttgaactctagtgaggagttgaagaggtttgttgagctcactaagattccagttgccagtactctaatgggtcttggatctcaccctctttctgatgatttgtctcttcaaatgcttggaatgcatggtactgtttatgcaaattatgctgttgataaggctgatttgttacttgcttttggagttaggtttgatgatcgtgtaacagggaagcttgaggcatttgctagtcgagctaagatagttcatattgatattgattcagctgagattggaaagaataagcaaccccatatatctgtttgtggtgatgttaaacctgctttggatggaatgaatcagatacttgagaagaacaaatcaaaacttaaacttgatttctcttcttggacaaaagagttggatgatcaaaaacagaaacaccctttgagtttcaagactttcggggaagcaattccacctcagtatgctattcaggttcttgatgaactgactgatgggaaagcaatcataagcactggtgttggtcaacatcaaatgtgggcagctcagttttacaagtacaacagacctcgtcaatggctgacatcaggtggattaggagctatgggttttggtttgcctgcagccattggagctgctgttgcaaacccagattcaatcgttgtagacatcgacggtgatgggagttttatcatgaatgttcaggagttggctaccattcgggtggagaatctccctgtgaagatactcctccttaacaatcaacacttgggcatggttgttcagtgggaggataggttctacaaggctaaccgagctcacacctacctcggagacccttctaaagaaactgagatattccccaacatgttgaagtttgctgatgcttgtggcataccagctgcacgtgtgacaaagaaggaagatcttagaggagccattcagaagatgttggacacacccggcccttatctcctggatgtaatagttcctcatcaagaacatgtcctgcctatgatcccaagtggtggagctttcaaagatgtgattacggaaggagatgggagaacaaagtactagagatatcagaggagtagcagttatgttttttccttactttctatgtacatttttgttctgtgttcttattaggccacgcttaaggcagtttatggattagagctagtagttatctttgctttttgcagccaacatgatgtactttcttatatctattaacataaaagtgttgtgtggcgactttatcaaattaggacatgtatttcaattttctggtatctaagctaagatccttgtaatactagtattcattgccctttttcttcaataatataaaatatgtcatttggtgtgctagggatta

SEQ ID NO: 2 - DNA, 2001 bp, Cannabis sativa
atggcggccgccaccactaatgctccaacctcctccttcctcacaaagacctctactttctcatcctcctccacctcctcttcaaaacccattctcagattcatactcccattctcctcatctccccataaccccgctctcttccgccgcccactccatctcaccaacacctccctccctcgccgaaaccctccttctgccgtaacaactccaacccccaccaccatttcctccgaccccgacaccttcgtctcccgattcggtcccgacgagccacgaaaaggcgcagatgttctcgtcgaagccctcgagcgcgaaggtgttaaagacgtcttcgcttacccaggtggtgcttccatggagatccaccaagctctgaccaggtctagcagcattcgaaatgtcctccctcgacacgagcagggtggtatctttgcggctgagggttacgctcgctcctctggtcttccaggtgtttgtattgcgacctccggccccggcgccaccaacttggtcagtggcttagccgacgcgctccttgatagtgttcctttagtcgccattactggacaagtcccaaggaggatgatcggtactgatgcgtttcaggaaacccctattgttgaggtaacaagatctattacaaagcataattaccttgttcttgatgtggaagacattcctagggtagttaaagaagctttctttttagccaaatcgggtcgacctggtcctgttttgattgatgtacctaaagatatacaacaacagcttgttgttcccaattgggatcaacccatgaagctacctgggtacttagctaggctccccaagctccctaatgaagctcatttggagcagattgtgaggctgatttctgagtcaaagaagccagttttgtatgttggtggtgggtgtttgaactctagtgaggagttgaagaggtttgttgagctcactaagattccagttgccagtactctaatgggtcttggatctcaccctctttctgatgatttgtctcttcaaatgcttggaatgcatggtactgtttatgcaaattatgctgttgataaggctgatttgttacttgcttttggagttaggtttgatgatcgtgtaacagggaagcttgaggcatttgctagtcgagctaagatagttcatattgatattgattcagctgagattggaaagaataagcaaccccatatatctgtttgtggtgatgttaaacctgctttggatggaatgaatcagatacttgagaagaacaaatcaaaacttaaacttgatttctcttcttggacaaaagagttggatgatcaaaaacagaaacaccctttgagtttcaagactttcggggaagcaattccacctcagtatgctattcaggttcttgatgaactgactgatgggaaagcaatcataagcactggtgttggtcaacatcaaatgtgggcagctcagttttacaagtacaacagacctcgtcaatggctgacatcaggtggattaggagctatgggttttggtttgcctgcagccattggagctgctgttgcaaacccagattcaatcgttgtagacatcgacggtgatgggagttttatcatgaatgttcaggagttggctaccattcgggtggagaatctccctgtgaagatactcctccttaacaatcaacacttgggcatggttgttcagtgggaggataggttctacaaggctaaccgagctcacacctacctcggagacccttctaaagaaactgagatattccccaacatgttgaagtttgctgatgcttgtggcataccagctgcacgtgtgacaaagaaggaagatcttagaggagccattcagaagatgttggacacacccggcccttatctcctggatgtaatagttcctcatcaagaacatgtcctgcctatgatcccaagtggtggagctttcaaagatgtgattacggaaggagatgggagaacaaagtactag

SEQ ID NO: 3 - protein, 666 aa, Cannabis sativa
MAAATTNAPTSSFLTKTSTFSSSSTSSSKPILRFILPFSSSPHNPALFRRPLHLTNTSLPRRNPPSAVTTPTPTTISSDPDTFVSRFGPDEPRKGADVLVEALEREGVKDVFAYPGGASMEIHQALTRSSSIRNVLPRHEQGGIFAAEGYARSSGLPGVCIATSGPGATNLVSGLADALLDSVPLVAITGQVPRRMIGTDAFQETPIVEVTRSITKHNYLVLDVEDIPRVVKEAFFLAKSGRPGPVLIDVPKDIQQQLVVPNWDQPMKLPGYLARLPKLPNEAHLEQIVRLISESKKPVLYVGGGCLNSSEELKRFVELTKIPVASTLMGLGSHPLSDDLSLQMLGMHGTVYANYAVDKADLLLAFGVRFDDRVTGKLEAFASRAKIVHIDIDSAEIGKNKQPHISVCGDVKPALDGMNQILEKNKSKLKLDFSSWTKELDDQKQKHPLSFKTFGEAIPPQYAIQVLDELTDGKAIISTGVGQHQMWAAQFYKYNRPRQWLTSGGLGAMGFGLPAAIGAAVANPDSIVVDIDGDGSFIMNVQELATIRVENLPVKILLLNNQHLGMVVQWEDRFYKANRAHTYLGDPSKETEIFPNMLKFADACGIPAARVTKKEDLRGAIQKMLDTPGPYLLDVIVPHQEHVLPMIPSGGAFKDVITEGDGRTKY

SEQ ID NO: 4 - DNA, 5337 bp, Cannabis sativa
aagctcaaaaattttcttgtacgttttctttccactttctctctctaaaaactcagactgtcggtggagtagtgaagagagagagagagagtttgagaccgtagttactctgtctctttcgtccccttgaagctatttttctcttttttctattttataatattttccttttctatatatctacagctatacaaagagagaagcgtttttagagagagagagagagagagagagagagagagagaaagagctgtgcttagcatagcatagcatagcagtagtctctctttgtgaccacttattttgttgatctgtgagatttgggcatctgggttttgtttcagataatcagatttgagatctggttgtgattttgatcaatttggctttggagctcaattgggtctaattacaaagctcagttttttgtttttcttaaggtaaagtttcaatctttattcatacttaatttcagtacaatatatgagtgttttagtgtacctaattccaactgtaaagataagatttttgatgtttatatgtatcttctttttcagttacagatctgggttttgaaagtgagagtatttggaggtatgcatagatctgattaagtcaatttgagctaccaatttgacccatttgagcttggtggcaatggaatcagaaggagaaactggggtaagtagtaacattgtactttcatgtttttattagtttgattagaaactatgatgttacatagtcaaaatactagaaacttaaaactttgatgatgttttagatttaggcaaattcatgattgtccttttgttattggatttggtttaactcagggaaatgccatgaagaatcttggtggtttggtctgtctgatatgtggtgatggtgttggaaagactgtggatggtaagccctttgttgcttgtgatgtatgcgattttccggtatgcagaccgtgctatgagtatgagcgaaaagacgggaatcaatcttgtcctcaatgcaaaaccagatataggaggcacaaaggtttggtttttagttgaattgggatatgaatttgatgttaatgtagtgaatgtgttggaatctgactttgtttttcgatctaatgcttgtttagggagtccggctatactcggggataaagaagaggatgttgatgttgatgatggtgggagtgatttcaattacccttcggaaaaccagagtgagcagcagaagattgcagagcgaatgttgagctggcaaatgacgtatggtcggggagaggatgttacggctcctaattatgataaagaggtttctcacaaccacataccattgctcaccaatggtcatgaggtgagtctatctaatctacaatatgtagtttcggtttttggaatgaaaagcctaatgtttttgaaatgttgtgtttaggtttcgggggaattgtcagctgcttctcctgagcggctttctatggcatctcctggacaagctggtgggaagcgtcaccattcctattcatccgatgttaatcaatcacgtatgtttgaaataatgaaatttaagaaaaaacagaacaaaacattttattgagattttgattgattcttgcagcaaacattagagttgtggatcccgtgagggagttcggttcacctggtatagggaacgtggcatggaaagagagagtggatggttggaagatgaagcaagagaagaatgttgttccaatgagcaccggccaagctaactccgaaagaggaggtggtgatatcgatgctagcactgatgtactcgtcgatgatactttactgtaagagtttatactcttcacttgatttagtaaaactactaaaattttctctttcacttgttgattcgcttttttatattattttttcagaaatgatgaagccaggcagcctctttcgaggaaggtttctattccttcatcgaggataaatccttataggatggttattgttcttcggcttgttattttatgcattttcttgcattaccggcttacaaatcccgtgccaaatgcatatgctctgtggttaatctctgtcatttgtgagatttggtttgctgtctcttggattctcgatcagtttcccaagtggcttcctgtaaaccgagaaacttatctcgacaggcttgctttaaggtaaaagacttaaatcgagtaactttgtcattttagattcgaagattatctgtctagagaaattgaaatctaattatttgtatttcaggtatgaccgagaaggtgaaacgtcacagttagctgcagttgacatctttgtcagtactgtcgacccattgaaagagccgccacttgtcacagccaacactgtgctatccattctcgctgttgactaccctgttgacaaggtttcttgctatgtttctgatgatggtgcagctatgctgtcttttgaatctctgtccgaaactgctgaattcgcaaggagatgggttcctttctgcaagaaatatagtatcgagcctcgtgcaccagaatggtacttcactcagaaaatcgattacttgaaagacaaggtccaaccgtcttttgttaaagagcgtagagctatgaagagagaatatgaagagttcaaagtccgagtcaatggacttgttgcaaaggcacagaaagttcccgaagaagggtggattatgcaagacggtactccatggcctggaaataacactagagatcacccgggaatgatccaagtaagattttcattcgtaaatttggaaaacctcgggagctacgaaagccttgttatgtaaaattgcttatttggtttgtttgtttgtttcaggttttcttgggacaaagcggaggacttgattctgatggcaacgagctacctcgattggtatatgtttctcgtgaaaagcgtcctggatttcaacatcacaagaaggctggtgctatgaatgcacttgtaagtatcgaaaaattcaaatgctaaagcaatttaattctttttgcaagttttcttacacgaaatgttcgattttattactctttgtaggttcgagtttcagcagtgcttaccaacggacccttcttgttgaatctcgattgtgatcattacattaacaacagcaaagccttgagagaagccatgtgtttcttaatggatcccaacttaggaaagaacgtttgttacgttcaattcccacagagattcgatggtattgataggaacgatagatatgccaatcgaaacactgtcttcttcgatgtaagttggttttttgtttattgttttactctttattttcggtttgtgttttaattctcactttgttggtttatttcagattaacttgcgaggtttggatggaattcaagggcccgtttatgtgggtacgggatgtgttttcaaccgaacagccttgtacggttatgaaccacccgttaagcctaagcataagaagactggcgtcatggcttcactatgctgtggttcgagaaagaagcgttcgaaatcaagtaaaaagggttctgataagaaaaaatctagcaagaatgctgatcctaccgtgccaattttcaatctcgaagatatagaagagggtgtggaaggtacacaattaatttcgatttactgtttttctctaaccatatctttgcaaatcggtctcattttctctttcacaattcaattcaggtgctggatttgatgatgagaaatcattgcttatgtcacaaatgagcctcgagaaacgattcggtcagtctgctgtttttgttgcctcaacgcttatggagaacggcggtgtacctcagtctgctactcccgaaactcttcttaaagaggctattcatgtcatcagttgcggatacgaagataaaacagattggggaaatgaggtttgtgacattttctgatcttcattccttttgcagacattttcgaaatttcttatttttttaacacaatcttgtgctggaacagattggatggatctacggttctgtcacagaagatattctcacgggattcaagatgcacgctcgtggttggaggtcgatttactgtatgcctaagcttgcagcctttaaagggtctgcacctatcaatctttccgatcgtttgaaccaagtgcttcgatgggctttaggttcagtcgaaattcttctcagtcggcattgtcctatttggtacggctacagtggtcggctcaaatggctcgaacggttcgcctatgtcaacaccaccatttatccaatcacttccattcctcttctcgcgtattgtacattgcccgccgtctgtcttctcacaaacaagttcatcattcctcaggttcgttcactcgacttatccacgaattcgtaataattgaaacttgtgcgtgttttgtcccacattgctcgtatgcaagtaatttgcggggtttatagggttttggtaattggataaaagctcgaaatctcaaaccctattgtttccgaaatgcagattagtaaccttgcaagtatatggtttatttccctctttctttccattttcgcaacgggtatcctcgagatgagatggagcggtgtagggatcgacgagtggtggagaaacgagcaattttgggtcatcggtggtgtttcagcccatctcttcgccgtggtccaaggtttgctcaaagtactagctggaatcgacacaaacttcactgtcacctcaaaggcatcagacgaagacggggatttcgccgagctttacttgttcaaatggacaactctgctcatcccgccaaccacgctcctcatcatcaacttggtgggagttgtggccggaatctcgtacgctattaacagcggttaccaatcgtggggacccttgttcggaaagttgtttttcgctttctgggtgattgttcatctctatcctttccttaaaggtctcatgggaagacagaacagaactcccaccattgttgtggtttggtctgttctacttgcttcaatcttttccttattgtgggttcgggttgacccgtttacgacccgagttaccggcccagatgttgagttgtgtggaatcaactgctagagatgaaaaaaattcaatctttattattttttagattatttattgttctaagggaaaaaaaaaagaacaacaaaagaaagctataaatcccataatatatgcaaatttgttttgcaatttcattttttttttggagatgtaatatgatgcttttttgtctttactgaactgaactggtttgtactccatgttaatcataaattatgtgcagataaaatagttgttaaagtttaaagattatattttgttgtatgaacatttttcttttgta

SEQ ID NO: 5 - DNA, 3243 bp, Cannabis sativa
atggaatcagaaggagaaactgggggaaatgccatgaagaatcttggtggtttggtctgtctgatatgtggtgatggtgttggaaagactgtggatggtaagccctttgttgcttgtgatgtatgcgattttccggtatgcagaccgtgctatgagtatgagcgaaaagacgggaatcaatcttgtcctcaatgcaaaaccagatataggaggcacaaagggagtccggctatactcggggataaagaagaggatgttgatgttgatgatggtgggagtgatttcaattacccttcggaaaaccagagtgagcagcagaagattgcagagcgaatgttgagctggcaaatgacgtatggtcggggagaggatgttacggctcctaattatgataaagaggtttctcacaaccacataccattgctcaccaatggtcatgaggtttcgggggaattgtcagctgcttctcctgagcggctttctatggcatctcctggacaagctggtgggaagcgtcaccattcctattcatccgatgttaatcaatcaccaaacattagagttgtggatcccgtgagggagttcggttcacctggtatagggaacgtggcatggaaagagagagtggatggttggaagatgaagcaagagaagaatgttgttccaatgagcaccggccaagctaactccgaaagaggaggtggtgatatcgatgctagcactgatgtactcgtcgatgatactttactaaatgatgaagccaggcagcctctttcgaggaaggtttctattccttcatcgaggataaatccttataggatggttattgttcttcggcttgttattttatgcattttcttgcattaccggcttacaaatcccgtgccaaatgcatatgctctgtggttaatctctgtcatttgtgagatttggtttgctgtctcttggattctcgatcagtttcccaagtggcttcctgtaaaccgagaaacttatctcgacaggcttgctttaaggtatgaccgagaaggtgaaacgtcacagttagctgcagttgacatctttgtcagtactgtcgacccattgaaagagccgccacttgtcacagccaacactgtgctatccattctcgctgttgactaccctgttgacaaggtttcttgctatgtttctgatgatggtgcagctatgctgtcttttgaatctctgtccgaaactgctgaattcgcaaggagatgggttcctttctgcaagaaatatagtatcgagcctcgtgcaccagaatggtacttcactcagaaaatcgattacttgaaagacaaggtccaaccgtcttttgttaaagagcgtagagctatgaagagagaatatgaagagttcaaagtccgagtcaatggacttgttgcaaaggcacagaaagttcccgaagaagggtggattatgcaagacggtactccatggcctggaaataacactagagatcacccgggaatgatccaagttttcttgggacaaagcggaggacttgattctgatggcaacgagctacctcgattggtatatgtttctcgtgaaaagcgtcctggatttcaacatcacaagaaggctggtgctatgaatgcacttgttcgagtttcagcagtgcttaccaacggacccttcttgttgaatctcgattgtgatcattacattaacaacagcaaagccttgagagaagccatgtgtttcttaatggatcccaacttaggaaagaacgtttgttacgttcaattcccacagagattcgatggtattgataggaacgatagatatgccaatcgaaacactgtcttcttcgatattaacttgcgaggtttggatggaattcaagggcccgtttatgtgggtacgggatgtgttttcaaccgaacagccttgtacggttatgaaccacccgttaagcctaagcataagaagactggcgtcatggcttcactatgctgtggttcgagaaagaagcgttcgaaatcaagtaaaaagggttctgataagaaaaaatctagcaagaatgctgatcctaccgtgccaattttcaatctcgaagatatagaagagggtgtggaaggtgctggatttgatgatgagaaatcattgcttatgtcacaaatgagcctcgagaaacgattcggtcagtctgctgtttttgttgcctcaacgcttatggagaacggcggtgtacctcagtctgctactcccgaaactcttcttaaagaggctattcatgtcatcagttgcggatacgaagataaaacagattggggaaatgagattggatggatctacggttctgtcacagaagatattctcacgggattcaagatgcacgctcgtggttggaggtcgatttactgtatgcctaagcttgcagcctttaaagggtctgcacctatcaatctttccgatcgtttgaaccaagtgcttcgatgggctttaggttcagtcgaaattcttctcagtcggcattgtcctatttggtacggctacagtggtcggctcaaatggctcgaacggttcgcctatgtcaacaccaccatttatccaatcacttccattcctcttctcgcgtattgtacattgcccgccgtctgtcttctcacaaacaagttcatcattcctcagattagtaaccttgcaagtatatggtttatttccctctttctttccattttcgcaacgggtatcctcgagatgagatggagcggtgtagggatcgacgagtggtggagaaacgagcaattttgggtcatcggtggtgtttcagcccatctcttcgccgtggtccaaggtttgctcaaagtactagctggaatcgacacaaacttcactgtcacctcaaaggcatcagacgaagacggggatttcgccgagctttacttgttcaaatggacaactctgctcatcccgccaaccacgctcctcatcatcaacttggtgggagttgtggccggaatctcgtacgctattaacagcggttaccaatcgtggggacccttgttcggaaagttgtttttcgctttctgggtgattgttcatctctatcctttccttaaaggtctcatgggaagacagaacagaactcccaccattgttgtggtttggtctgttctacttgcttcaatcttttccttattgtgggttcgggttgacccgtttacgacccgagttaccggcccagatgttgagttgtgtggaatcaactgctag

SEQ ID NO: 6 - protein, 1080 aa, Cannabis sativa
MESEGETGGNAMKNLGGLVCLICGDGVGKTVDGKPFVACDVCDFPVCRPCYEYERKDGNQSCPQCKTRYRRHKGSPAILGDKEEDVDVDDGGSDFNYPSENQSEQQKIAERMLSWQMTYGRGEDVTAPNYDKEVSHNHIPLLTNGHEVSGELSAASPERLSMASPGQAGGKRHHSYSSDVNQSPNIRVVDPVREFGSPGIGNVAWKERVDGWKMKQEKNVVPMSTGQANSERGGGDIDASTDVLVDDTLLNDEARQPLSRKVSIPSSRINPYRMVIVLRLVILCIFLHYRLTNPVPNAYALWLISVICEIWFAVSWILDQFPKWLPVNRETYLDRLALRYDREGETSQLAAVDIFVSTVDPLKEPPLVTANTVLSILAVDYPVDKVSCYVSDDGAAMLSFESLSETAEFARRWVPFCKKYSIEPRAPEWYFTQKIDYLKDKVQPSFVKERRAMKREYEEFKVRVNGLVAKAQKVPEEGWIMQDGTPWPGNNTRDHPGMIQVFLGQSGGLDSDGNELPRLVYVSREKRPGFQHHKKAGAMNALVRVSAVLTNGPFLLNLDCDHYINNSKALREAMCFLMDPNLGKNVCYVQFPQRFDGIDRNDRYANRNTVFFDINLRGLDGIQGPVYVGTGCVFNRTALYGYEPPVKPKHKKTGVMASLCCGSRKKRSKSSKKGSDKKKSSKNADPTVPIFNLEDIEEGVEGAGFDDEKSLLMSQMSLEKRFGQSAVFVASTLMENGGVPQSATPETLLKEAIHVISCGYEDKTDWGNEIGWIYGSVTEDILTGFKMHARGWRSIYCMPKLAAFKGSAPINLSDRLNQVLRWALGSVEILLSRHCPIWYGYSGRLKWLERFAYVNTTIYPITSIPLLAYCTLPAVCLLTNKFIIPQISNLASIWFISLFLSIFATGILEMRWSGVGIDEWWRNEQFWVIGGVSAHLFAVVQGLLKVLAGIDTNFTVTSKASDEDGDFAELYLFKWTTLLIPPTTLLIINLVGVVAGISYAINSGYQSWGPLFGKLFFAFWVIVHLYPFLKGLMGRQNRTPTIVVVWSVLLASIFSLLWVRVDPFTTRVTGPDVELCGINC

SEQ ID NO: 7 - DNA, 4716 bp, Cannabis sativa
ctcagttaaacagaacaagttgacaaggggagaagagagaaggcaaaattgtgtattctagaattttatgttgcttttctgtgtgcccaagaaaaatgttaaacgaagacttaccatatgagatgagtcggtttcttttaatgaaataagactacacgatcacagcaactcttataataaagcatattaaggatggtgcttctgctagaaacctatacattgacaaaagttctctctacactcaagctcatatagcagatatagaatttgccccactggcttattagaagggtgaaaaaatagcatgaagggtttgcagaagttacaacaaataactacgattacagtcttaataataatgagaagctcaacttgcaatacagtcgaccaaaatcaaacacatgataattgaaaaggattgccttgcctcccaactgctttagttgtaatgacagtagccgatgctattgttctctatccagttaataaatactcattcagtgctttgtaaatctctcaaggacttcaaagtaatctgggaaagtttttcgggtgcaaccaggatccttaatggtaactggcacatctgaacaagctgcaagagagaacgccatagccatcctgtggtcgtcgtatgtgtctattgctgtgatatttagtttctctggtggagtgatcacgcagtaatcgggtccttcctcaactgttgctccaagctaaaatttttgtatccataagaacagatagctatcagccattacattccaaggcgcatgatataacaagtacataaaacagcataaaaactaacctttctgagttcagtgcagatggcaatcattctctctgtctccttgactctccaacttgccactgggaattaggaaaggcaagaaatgagtacggtatctaaccctctaaacaaaataacattttattggttttcatttttttctctaaaatttattgcaaatgagcagattaatttttattatggcagacgagaaccgatgatctaccccaacccaaaccgactgccccattttaccatctcacaaaattaaggaaaaacataccatctcttatagcagtggggccatcagcaaaaagagcaactacagcaagagtcatggcaacatcaggcattttgttcatgttgacatcaatggcttttaagtgtttactttttacagaatctcgtggtggtccagtgacagtgacactgttctctgtccagctaactttagcacccattttctcaagaacttcagcaaatttaacgtctccctttatgaaacaccagttaatgtatgtaaatacacttaaatcttagattctatgtaaacacaagtacaagaagaagagatttcaaggtctaagcaaaatacctgtaaactactagtcccacaaccttctacggtgacagtaccaccagtgactgcagcaccagctaggaagtaactagcacttgaagcatcaccttcaacataagcgtttccaggagatctgacagatttttattttgcaatttattactctttgacataaccagatgtaatgtaacattactatcaactatattcagaagaaacctacttgtacttttgacctcctttaactaaaaatcgatcccagctatcactgtgttcaacagtaaccccaaaccgtgccatcaacttcaaagtcatatcaacatagggaaccgagatcaatttatcaattatctcaatttcaacatctccaagagccaagggagctgccataagcaaggctgtcaaatattgactgctaattgatccagaaagtttcacctgcaaagggtaatattacataattctgagtcaaaatgatgaagtttgtcatcatttaagatatattaaatgctgtagattaatcactacttatgcatcatttgacaatattgtttttatttaaaagcaaaacaacctaaaaataaaccagtcaatggatttcataacataaaaccactattacaacgatgttttatggccaaaaagaataagaagaagcaatgcaagcctcaccttgcccccaggaaggcctccttttccaagcacacgaacagggggacaatccgtaccatgaaaacaatcaacatctgcaccaagctgcttaagaccagtcaccaaatctccaatcggtctttctctcattcgaggaacaccatcaagtacgtaactgagatatagaagttaagaggttactgagaaaatgtaataacagcatacaaaataatttatgatatgtgctagttcatttccaacatgtaatttctagcataaccacatcaaagtaaagagggaagtataaatggtgatgttgttagcaaaagtgactttctttactcaaaatgtaagagctactcggccagttaagaaaaacaagagcataaattcactgcattagaagtactttatattcaaaagcaattgcaatgaagacaaacctagcatttccaccagcaacagtcaccgcagctgtgagtggacgcattgctgttccagcatttccaaggaaaagttgaacttcgtcaacagattctttacctgcaggaaactgacccccacaaccttccacaatcgctcgcttgctttccttgtctgcttcaacacgaagaccaagggtttccaaggcaccaagcatgtagtgaatgtcatcactatctaacaagttgtccacaacagttgtcccctgaataattataacaaaaaaaaaacagttacttcccaccgctcaaactattttatagggcaaaaaactcaagtacatgaaaataacatgaactgtaaagataagtagtagcgccagaatatggggaagaaatagagaaagagtatatggagaatgaacagctacaaattctagatgactcaaacactaatagtataacttcattctaaatcccacattctaaaatttagggagttcaggtgagacaatgcaccaacgaaacacctcatatataatgtaacagaagcgtagagttatgaaattctcataaataattaacaaaataaactgcaaggagtccaaatctctgcagtgtggagctctagcctaaagttgtaaaagcacaactgagaccagaaatataacttcagtaacccagttgagttgcattttttatttccatcatattctaacgaatttaaccaaaatatcatattactttactccatttctctcaatttccctcccaattaagggcatgagttcagcttcactgaacactattcattttcaccaataaattgaaaatgctaagctttactctccattttcttgaaaaaaaaaaacattccatcgctgcaaactcttaacataaaactaaaagtaaattgctcacaaaaagcacaatctttgtatacctcaaaaatacttttcaaatttgtggataacaaaggaagaattccatgcaattacatggtttaccagttgggcagtcgatgctgatcatggttcagatcttgctaatgtaactaactacagtttccagccacacgatccattggaactaattttagtgggaatgaggaaaattacaaggaaaatgaaagcaaatgacaaaataattacgatatagtattttatagctgtggagcttgagataggaaagtagagtatggcatagcatgacatgatttgagagcttagcctagttttggttcattttcgtattgatgttccatatttttactctcttacctttgtatatacagaaaacttcctcgacaaaaataaatgtttgaagttttaaaaaattgaaatatacctcagaaagagcagccaggagcagaatccgattggatagtgacttggaacccggcaacttaacagtgccagagatatctttaatgggctgcaacacaatctccggtacctttgaaggcttctccgctgtgacagctgaagccatgactggaccagccatagctttaccaaccgtagaacaacccccaaattcagttccaatcttcaaactcaaagagttagatgaacccaaaaacggtgatctcaacgaaactgaatttagggatcttggtgtatgagacttagaaatatttgggagagtaaggatagtttgagctccattgctacagattttgctcacttgggccatttttttttcttctctctctctctgaaacccaaagcttctctgactctctctatacaaataaaggttggatttttggtggcaatggtaacgttaatggatgctgctgctgtaggtcgacattgactctctttcaattttcaaagagggtaggagggcttaccaaccatccaagcccaaaatcgtctctttcaccaagaaatttgggccaaaatgttgaatttccaaaacttagtcaccttaatttaatatccaaaaagttaagggacccatttgaccggtagtagttggtggtctttgatgcaagtctgtgcttgtacgttgtgctaacaatgtagtactctatagtagttttggggtataaaagaaaagaaaagaaaagttgatcttggggttttttgggaaaaagtggtgagtctaattctttcaccaacccattacaatttagtggggtcgaagccaccaaagggggtaggtaggtctaatacacttgcactgcatttgcaattttaatatgcattcttatttcttaaagttattatataataaaataaattatttcacaataataatgatct

SEQ ID NO: 8 - DNA, 1534 bp, Cannabis sativa
atggcccaagtgagcaaaatctgtagcaatggagctcaaactatccttactctcccaaatatttctaagtctcatacaccaagatccctaaattcagtttcgttgagatcaccgtttttgggttcatctaactctttgagtttgaagattggaactgaatttgggggttgttctacggttggtaaagctatggctggtccagtcatggcttcagctgtcacagcggagaagccttcaaaggtaccggagattgtgttgcagcccattaaagatatctctggcactgttaagttgccgggttccaagtcactatccaatcggattctgctcctggctgctctttctgaggggacaactgttgtggacaacttgttagatagtgatgacattcactacatgcttggtgccttggaaacccttggtcttcgtgttgaagcagacaaggaaagcaagcgagcgattgtggaaggttgtgggggtcagtttcctgcaggtaaagaatctgttgacgaagttcaacttttccttggaaatgctggaacagcaatgcgtccactcacagctgcggtgactgttgctggtggaaatgctagttacgtacttgatggtgttcctcgaatgagagaaagaccgattggagatttggtgactggtcttaagcagcttggtgcagatgttgattgttttcatggtacggattgtccccctgttcgtgtgcttggaaaaggaggccttcctgggggccaggtgaaactttctggatcaattagcagtcaatatttgacagccttgcttatggcagctcccttggctcttggagatgttgaaattgagataattgataaattgatctcggttccctatgttgatatgactttgaagttgatggcacggtttggggttactgttgaacacagtgatagctgggatcgatttttagttaaaggaggtcaaaagtacagatctcctggaaacgcttatgttgaaggtgatgcttcaagtgctagttacttcctagctggtgctgcagtcactggtggtactgtcaccgtagaaggttgtgggactagtagtttaaagggagacgttaaatttgctgaagttcttgagaaaatgggtgctaaagttagctggacagagaacagtgtcactgtcactggaccaccacgagattctgtaaaaagtaaacacttaaaagccattgatgtcaacatgaacaaaatgcctgatgttgccatgactcttgctgtagttgctctttttgctgatggccccactgctataagagatgtggcaagttggagagtcaaggagacagagagaatgattgccatctgcactgaactcagaaagcttggagcaacagttgaggaaggacccgattactgcgtgatcactccaccagagaaactaaatatcacagcaatagacacatacgacgaccacaggatggctatggcgttctctcttgcagcttgttcagatgtgccagttaccattaaggatcctggttgcacccgaaaaactttcccag

SEQ ID NO: 9 - protein, 511 aa, Cannabis sativa
MAQVSKICSNGAQTILTLPNISKSHTPRSLNSVSLRSPFLGSSNSLSLKIGTEFGGCSTVGKAMAGPVMASAVTAEKPSKVPEIVLQPIKDISGTVKLPGSKSLSNRILLLAALSEGTTVVDNLLDSDDIHYMLGALETLGLRVEADKESKRAIVEGCGGQFPAGKESVDEVQLFLGNAGTAMRPLTAAVTVAGGNASYVLDGVPRMRERPIGDLVTGLKQLGADVDCFHGTDCPPVRVLGKGGLPGGQVKLSGSISSQYLTALLMAAPLALGDVEIEIIDKLISVPYVDMTLKLMARFGVTVEHSDSWDRFLVKGGQKYRSPGNAYVEGDASSASYFLAGAAVTGGTVTVEGCGTSSLKGDVKFAEVLEKMGAKVSWTENSVTVTGPPRDSVKSKHLKAIDVNMNKMPDVAMTLAVVALFADGPTAIRDVASWRVKETERMIAICTELRKLGATVEEGPDYCVITPPEKLNITAIDTYDDHRMAMAFSLAACSDVPVTIKDPGCTRKTFP

SEQ ID NO: 10 - DNA, 4309 bp, Cannabis sativa
aaaaataaataaagagagagagaaaccctatgagatgtacagaccattcgcactctctctcgtctctctatcgtatctaaaacccatcttcatcttcttcattttcttcttctccctttctcaagatccaacgtcgacggctccactctgactgactggtattctcatcagttcttaacattttctaaatctttattttgcttttaggttaataattttagggttttttatattttatatttttttagggcttttgatatcttctagtaaggcgacaatggcgtccacagacccagagatagcgaagactcaggaagaacgcaggaagatggagcagcagcttgcttcgcttaactctgttactttcgatacggatctctatggtggaaatgatagggatgcttatgtctcatccattcctgtcaccgacgatgatgagaacaacgaccctatggacaatgaggttgtgcgcaagttagcttcttacactgctcctaagtcgctcataaatgatgtccccaggggaggagacgatgatgctggtttggggcctaagaaacccaattcgattttgcaacgagaggatgcatatcgtcagaggaggttgaaccaggtgatttcgcctgatcgaaatgatccttttgcatcgggggagatgacaccggacccctcggtgaggacctatgctgatgtcatgaaggaattggccttaaagaagaaggagcaggagactcttaggcttatttcggagaagaagaaggatcaagaagaggccgctaaggcagcggctgagaggggtgagactgcccctgtggctgctggtgctccccagaagaggagaaataggtgggacctttctcaggatgatggttctgcaaagaagcccaagacttccgattgggatttgcctgatacgacaccaaaccggtgggacacaactccaactccagggagacttggggatgcgactcctggtgctggtgctgggaggaggaatcggtgggatgaaactcccacgcctgggcggttggtggattcagatgccactccggctggtgctgtcacccctggtgctacaccttctgggatgtcttgggattcaacccctaagcttggtgggctagctacgcccactcccaagaagcagaggtctaggtgggatgagacaccggcaacaatgggaagtgccacacctggagctacaccagcagctgcttatacacccggtgtgaccccggtgggagcagctgaacttgctacgccaacgccaggggctattaatttgcggggttcaattactccagaacagtataacttgttgagatgggagaaagatatcgaggaaaggaaccgtcccttaaccgatgaagagcttgattctatgtttccacaagagggatacaagattttggaccctccagcttcatacgtacctattaggacacctgcgagaaagcttcttgctaccccaacaccgatggcaactcctctttattccatcccagaagagaatcgtgggcagcaatttgatgttccaaaggaatctgcaggtggcttgccatttatgaagccagaggattaccagtattttggggctttgttgaatgaggatgatgaggaagaattgtctcctgatgaacagaaggagcgtaaaattatgaagcttctgctcaaggttaagaatggtacacctccacagaggaagacagcattgagacagcttactgataaagctcgagagtttggtgccggtccattgtttaaccgcattctgccattgcttatgcaacccactttggaagaccaagagagacatcttttggtcaaggttattgatagagtgctgtataaattggacgaattggtgagaccctacgtgcacaaaattcttgtcgtcattgagcctttgttgattgatgaagactactatgctcgtgtggaagggagagagattatctccaaccttagtaaagcagctggactggctactatgattgctgccatgcgtccagatattgacaacattgatgaatatgttagaaatacaactgctagagcttttagtgttgttgcttctgcccttggtattcctgctctgttacctttcttgaaagctgtttgtcagagtaagaaatcgtggcaagctcgacatactggaattaagattgtgcagcagattgctatcttgattggttgtgctgttcttccacatttgaggtctcttgttgaaattattgagcacgggctcaatgacgagaatcaaaaagtgaggactattactgcattgtcacttgctgcacttgctgaggctgccgctccttatggtattgagagctttgactctgttctaaagcccttgtggaagggtattagatcacatcgtggtaaggtcttggctgcatttttgaaggccatcggttttattattcctttgatggatgccatttatgccagttactataccaaggaagtgatggtaatattgattcgagagttccagtcacctgatgaagaaatgaagaaaatagtgctgaaagtggtgaagcagtgtgtgagtacagagggtgttgaggctgattatatcagaaatgatattcttccggagttctttaaaaacttttgggttaggagaatggcattagataggagaaattataggcaactagtggaaacaactgtagagatggcaaacaaagttggtgttgctgatattgtcggacgaattgttgaggatctaaaagatgagagtgaaccatatagacgaatggttatggagacaattgaaaaggtagttgcaaatctgggtgcatctgatatcgaccctcgattggaggagcttttaatcgatggaattctttatgctttccaagagcagaccagtgatgatgctaatgttatgcttaatgggtttggagcagtggtaaattctcttggacagagagtccaaccctatttgcctcagatttgtggtactatcaagtggcgtttgaataacaagagtgcaaaggtgagacagcaagctgcggatcttatttcaaggattgctgttgtcatgaagcagtgccaagaggaacaacttatgggccatcttggtgttgtactgtacgagtacttgggagaagaataccctgaagttcttggatcaatcttgggagcattgaaagcaattgtcaatgtcattggtatgacaaagatgacacctcctattaaagatttgcttcctagattgactccaattttgaagaacaggcacgagaaagtccaagaaaactgtattgaccttgtaggtcgaattgctgaccgtggtgcagagtttgtcccagctagggagtggatgaggatttgttttgaacttcttgagatgctcaaggcccacaaaaagggtatccggcgagctactgtgaacacatttggctacattgcaaaagccatcggtccacaggatgtcctggcaactttattgaacaatctgaaggttcaggaacgtcaaaaccgtgtctgcaccactgtagctatcgccatagttgcagaaacttgttctcctttcacagttttgccagctctgatgaacgagtaccgtgttcctgagctgaacgttcagaatggtgtcctgaaatcgctatctttcctgtttgagtacattggcgaaatgggaaaagactacatatacgcagttactccattgctggaggatgctctcatggacagagatctggttcatcggcagacagcagcttcagctgtcaagcacatggctttgggagtggctggtttgggttgcgaggacgcgttggttcacttgctgaattatgtgtggccgaacatattcgagacctcgcctcacgtcattaatgccgtcatggaagccattgaaggcatgagggtggccttgggagctgcggttgtgctaaactactgtctccagggtctctttcatcctgctcgaaaggtcagagaagtttactggaagatttataactcgttgtatattggagctcaagatgcccttgtagcagcttatccagttctggacgatgagcaaaataacatatatagtaggcctgagttggttatgtttctgtgaattgtagcctgtttgtagttagagaggacaatagaattctacgagaacagttttagtttaaaaaaaaatggaaaatatttcaagagactctgttgtatttacttttaggtactttggtttttctgatactttcaaatggggaaagagggcctgtgagccattaattgtgtttttgaatctgtgtttgagtttccatatatgttatattattgttctctttatcta

SEQ ID NO: 11 - DNA, 3807 bp, Cannabis sativa
atggcgtccacagacccagagatagcgaagactcaggaagaacgcaggaagatggagcagcagcttgcttcgcttaactctgttactttcgatacggatctctatggtggaaatgatagggatgcttatgtctcatccattcctgtcaccgacgatgatgagaacaacgaccctatggacaatgaggttgtgcgcaagttagcttcttacactgctcctaagtcgctcataaatgatgtccccaggggaggagacgatgatgctggtttggggcctaagaaacccaattcgattttgcaacgagaggatgcatatcgtcagaggaggttgaaccaggtgatttcgcctgatcgaaatgatccttttgcatcgggggagatgacaccggacccctcggtgaggacctatgctgatgtcatgaaggaattggccttaaagaagaaggagcaggagactcttaggcttatttcggagaagaagaaggatcaagaagaggccgctaaggcagcggctgagaggggtgagactgcccctgtggctgctggtgctccccagaagaggagaaataggtgggacctttctcaggatgatggttctgcaaagaagcccaagacttccgattgggatttgcctgatacgacaccaaaccggtgggacacaactccaactccagggagacttggggatgcgactcctggtgctggtgctgggaggaggaatcggtgggatgaaactcccacgcctgggcggttggtggattcagatgccactccggctggtgctgtcacccctggtgctacaccttctgggatgtcttgggattcaacccctaagcttggtgggctagctacgcccactcccaagaagcagaggtctaggtgggatgagacaccggcaacaatgggaagtgccacacctggagctacaccagcagctgcttatacacccggtgtgaccccggtgggagcagctgaacttgctacgccaacgccaggggctattaatttgcggggttcaattactccagaacagtataacttgttgagatgggagaaagatatcgaggaaaggaaccgtcccttaaccgatgaagagcttgattctatgtttccacaagagggatacaagattttggaccctccagcttcatacgtacctattaggacacctgcgagaaagcttcttgctaccccaacaccgatggcaactcctctttattccatcccagaagagaatcgtgggcagcaatttgatgttccaaaggaatctgcaggtggcttgccatttatgaagccagaggattaccagtattttggggctttgttgaatgaggatgatgaggaagaattgtctcctgatgaacagaaggagcgtaaaattatgaagcttctgctcaaggttaagaatggtacacctccacagaggaagacagcattgagacagcttactgataaagctcgagagtttggtgccggtccattgtttaaccgcattctgccattgcttatgcaacccactttggaagaccaagagagacatcttttggtcaaggttattgatagagtgctgtataaattggacgaattggtgagaccctacgtgcacaaaattcttgtcgtcattgagcctttgttgattgatgaagactactatgctcgtgtggaagggagagagattatctccaaccttagtaaagcagctggactggctactatgattgctgccatgcgtccagatattgacaacattgatgaatatgttagaaatacaactgctagagcttttagtgttgttgcttctgcccttggtattcctgctctgttacctttcttgaaagctgtttgtcagagtaagaaatcgtggcaagctcgacatactggaattaagattgtgcagcagattgctatcttgattggttgtgctgttcttccacatttgaggtctcttgttgaaattattgagcacgggctcaatgacgagaatcaaaaagtgaggactattactgcattgtcacttgctgcacttgctgaggctgccgctccttatggtattgagagctttgactctgttctaaagcccttgtggaagggtattagatcacatcgtggtaaggtcttggctgcatttttgaaggccatcggttttattattcctttgatggatgccatttatgccagttactataccaaggaagtgatggtaatattgattcgagagttccagtcacctgatgaagaaatgaagaaaatagtgctgaaagtggtgaagcagtgtgtgagtacagagggtgttgaggctgattatatcagaaatgatattcttccggagttctttaaaaacttttgggttaggagaatggcattagataggagaaattataggcaactagtggaaacaactgtagagatggcaaacaaagttggtgttgctgatattgtcggacgaattgttgaggatctaaaagatgagagtgaaccatatagacgaatggttatggagacaattgaaaaggtagttgcaaatctgggtgcatctgatatcgaccctcgattggaggagcttttaatcgatggaattctttatgctttccaagagcagaccagtgatgatgctaatgttatgcttaatgggtttggagcagtggtaaattctcttggacagagagtccaaccctatttgcctcagatttgtggtactatcaagtggcgtttgaataacaagagtgcaaaggtgagacagcaagctgcggatcttatttcaaggattgctgttgtcatgaagcagtgccaagaggaacaacttatgggccatcttggtgttgtactgtacgagtacttgggagaagaataccctgaagttcttggatcaatcttgggagcattgaaagcaattgtcaatgtcattggtatgacaaagatgacacctcctattaaagatttgcttcctagattgactccaattttgaagaacaggcacgagaaagtccaagaaaactgtattgaccttgtaggtcgaattgctgaccgtggtgcagagtttgtcccagctagggagtggatgaggatttgttttgaacttcttgagatgctcaaggcccacaaaaagggtatccggcgagctactgtgaacacatttggctacattgcaaaagccatcggtccacaggatgtcctggcaactttattgaacaatctgaaggttcaggaacgtcaaaaccgtgtctgcaccactgtagctatcgccatagttgcagaaacttgttctcctttcacagttttgccagctctgatgaacgagtaccgtgttcctgagctgaacgttcagaatggtgtcctgaaatcgctatctttcctgtttgagtacattggcgaaatgggaaaagactacatatacgcagttactccattgctggaggatgctctcatggacagagatctggttcatcggcagacagcagcttcagctgtcaagcacatggctttgggagtggctggtttgggttgcgaggacgcgttggttcacttgctgaattatgtgtggccgaacatattcgagacctcgcctcacgtcattaatgccgtcatggaagccattgaaggcatgagggtggccttgggagctgcggttgtgctaaactactgtctccagggtctctttcatcctgctcgaaaggtcagagaagtttactggaagatttataactcgttgtatattggagctcaagatgcccttgtagcagcttatccagttctggacgatgagcaaaataacatatatagtaggcctgagttggttatgtttctgtga

SEQ ID NO: 12 - protein, 1268 aa, Cannabis sativa
MASTDPEIAKTQEERRKMEQQLASLNSVTFDTDLYGGNDRDAYVSSIPVTDDDENNDPMDNEVVRKLASYTAPKSLINDVPRGGDDDAGLGPKKPNSILQREDAYRQRRLNQVISPDRNDPFASGEMTPDPSVRTYADVMKELALKKKEQETLRLISEKKKDQEEAAKAAAERGETAPVAAGAPQKRRNRWDLSQDDGSAKKPKTSDWDLPDTTPNRWDTTPTPGRLGDATPGAGAGRRNRWDETPTPGRLVDSDATPAGAVTPGATPSGMSWDSTPKLGGLATPTPKKQRSRWDETPATMGSATPGATPAAAYTPGVTPVGAAELATPTPGAINLRGSITPEQYNLLRWEKDIEERNRPLTDEELDSMFPQEGYKILDPPASYVPIRTPARKLLATPTPMATPLYSIPEENRGQQFDVPKESAGGLPFMKPEDYQYFGALLNEDDEEELSPDEQKERKIMKLLLKVKNGTPPQRKTALRQLTDKAREFGAGPLFNRILPLLMQPTLEDQERHLLVKVIDRVLYKLDELVRPYVHKILVVIEPLLIDEDYYARVEGREIISNLSKAAGLATMIAAMRPDIDNIDEYVRNTTARAFSVVASALGIPALLPFLKAVCQSKKSWQARHTGIKIVQQIAILIGCAVLPHLRSLVEIIEHGLNDENQKVRTITALSLAALAEAAAPYGIESFDSVLKPLWKGIRSHRGKVLAAFLKAIGFIIPLMDAIYASYYTKEVMVILIREFQSPDEEMKKIVLKVVKQCVSTEGVEADYIRNDILPEFFKNFWVRRMALDRRNYRQLVETTVEMANKVGVADIVGRIVEDLKDESEPYRRMVMETIEKVVANLGASDIDPRLEELLIDGILYAFQEQTSDDANVMLNGFGAVVNSLGQRVQPYLPQICGTIKWRLNNKSAKVRQQAADLISRIAVVMKQCQEEQLMGHLGVVLYEYLGEEYPEVLGSILGALKAIVNVIGMTKMTPPIKDLLPRLTPILKNRHEKVQENCIDLVGRIADRGAEFVPAREWMRICFELLEMLKAHKKGIRRATVNTFGYIAKAIGPQDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTAASAVKHMALGVAGLGCEDALVHLLNYVWPNIFETSPHVINAVMEAIEGMRVALGAAVVLNYCLQGLFHPARKVREVYWKIYNSLYIGAQDALVAAYPVLDDEQNNIYSRPELVMFL

SEQ ID NO: 13 - protein, 1379 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 14 - protein, 1379 aa, Streptococcus pyogenes
  MUTAGEN (10)..(10)
  MUTAGEN (840)..(840)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

(19) United States
(12) Patent Application Publication
(10) Pub. No.: US 2024/0309394 A1
(43) Pub. Date: Sep. 19, 2024

(54) HERBICIDE RESISTANT CANNABIS PLANT

(71) Applicant: BETTERSEEDS LTD, Givat Chen (IL)

(72) Inventors: Tal SHERMAN, Rehovot (IL); Ido MARGALIT, Gan Yavne (IL); Shira COREM, Sde Hemed (IL)

(73) Assignee: BETTERSEEDS LTD, Givat Chen (IL)

(21) Appl. No.: 18/270,366

(22) PCT Filed: Dec. 26, 2021
(86) PCT No.: PCT/IL2021/051534

§ 371 Date: Jun. 29, 2023

Related U.S. Application Data
(60) Provisional application No. 63/131,912, filed on Dec. 30, 2020.

(51) Int. Cl. C12N 15/82; C12N 9/10; C12N 9/22; C12N 9/78; C12N 15/11; C12Q 1/6895

(52) U.S. Cl. CPC C12N 15/8274; C12N 9/1022; C12N 9/1059; C12N 9/1092; C12N 9/22; C12N 9/78; C12N 15/111; C12N 15/8213; C12Q 1/6895; C12Y 202/01006; C12Y 204/01012; C12Y 205/01019; C07K 2319/00; C12N 2310/20; C12N 2800/80; C12Q 2600/13; C12Q 2600/156

(57) ABSTRACT

The present invention discloses a modified Cannabis plant exhibiting herbicide resistance (HR) as compared to a Cannabis plant absent of such modification. The modified plant comprises at least one genetically modified HR-related gene comprising at least one mutation conferring herbicide resistance to the plant. The present invention further discloses methods for producing the same.

### FIELD OF THE INVENTION

The present disclosure relates to Cannabis plants with improved traits. More particularly, the current invention pertains to producing herbicide resistant Cannabis plants by manipulating genes using genome editing techniques.

### BACKGROUND OF THE INVENTION

The Cannabis market is enjoying an unprecedented spike in activity following the wide spread legalization trend across the world. It is estimated that the American market alone would reach a value of at least $30B by 2025, with an exceptional growth rate of 30% per annum. This has led to an increase in demand for Cannabis products for medicinal or recreational use. To meet the demand, a growing need for the implementation of new and advanced breeding and cultivation techniques has become apparent in Cannabis breeding programs.

Herbicide resistance is one of the most important traits for plant biotechnology, which is widely used to improve agricultural efficiency by controlling weeds. For these reasons, new technologies for conferring herbicide resistance to plants are continuously developing.

Previously, traditional plant breeding techniques have been widely used to improve various crops. However, it is labor intensive and also takes time to progress from screening phenotypes and genotypes to crosses for the development of commercial varieties. Furthermore, the probability of obtaining herbicide-resistant mutations (especially multiple mutations in a same gene) by traditional breeding is very low, and it is possible to produce linked undesired mutations. Later, genetic modification (GM) technology has been used to develop value-added crops with beneficial traits by the transfer of gene(s) into elite varieties. However, there are health and environmental safety concerns on the developed genetically modified crops, requiring significant time and costs for the regulation approval process to be commercialized. Moreover, the integration of transgenes into the plant genome is non-specific and sometimes unstable in genetically modified crops. In addition, transgenic technology can only introduce known herbicide-resistant genes into the plant of interest to confer the expected herbicide resistance.

Herbicides target key enzymes in the plant metabolic pathway, which disrupt plant food production and eventually kill it. Genetically modified (GM) herbicide-resistant (HR) crops have been cultivated by farmers throughout the world, especially in the United States.

Glyphosate and glufosinate herbicides are useful for weed control and have minimal direct impact on animal life, and are not persistent. They are highly effective and among the safest of agrochemicals to use. Glyphosate herbicide kills plants by blocking the EPSPS enzyme, an enzyme involved in the biosynthesis of aromatic amino acids, vitamins and many secondary plant metabolites. There are several ways by which crops can be modified to be glyphosate-tolerant. One commonly used strategy to produce a glyphosate-tolerant plant is to incorporate a soil bacterium gene that produces a glyphosate tolerant form of EPSPS. Another used way is to incorporate a different soil bacterium gene that produces a glyphosate degrading enzyme. Glufosinate herbicides contain the active ingredient phosphinothricin, which kills plants by blocking the enzyme responsible for nitrogen metabolism and for detoxifying ammonia, a by-product of plant metabolism. Crops modified to tolerate glufosinate contain a bacterial gene that produces an enzyme that detoxifies phosphinothricin and prevents it from doing damage.

However, genetically-Engineered (GE) crops have been considered as a cause for increased HR-weeds development. There are many risks associated with the production of GM herbicide-resistant crops, including grain contamination, segregation and introgression of herbicide-resistant traits and marketplace acceptance. These drawbacks are represented in the occurrence of weed population shifts, the evolution of herbicide-resistant weed populations and herbicide-resistant crops becoming volunteer weeds. Examples of herbicide-resistant weeds include populations of horseweed (Conyza canadensis (L) Cronq) resistant to N-(phosphonomethyl) glycine (glyphosate).

Thus GM-HRCs are often referred to as ‘first generation crops’ and questions have been raised as to their usefulness and putative risks to the environment and to consumers.

Therefore, a more precise breeding technique is highly required. Plant genome editing technology using engineered nucleases might open possibilities to accelerate plant breeding for desirable traits. CRISPR/Cas-mediated gene editing methods enable precise modifications of DNA sequences and offer a great promise for the improvement of crops.

Currently available herbicide-resistant crops developed for commercialization include maize, soybean, canola, cotton, rice, tobacco, sugar beet (adapted from GM Approval Database of ISAAA (https://www.isaaa.org/gmapp roval datab ase). The HR crops have been developed by introducing genes that can confer resistance specific to different kinds of herbicides. Initial attempts to use CRISPR/Cas9-mediated gene editing for developing HR plants have been applied to soybean, maize, rice, flax, chili pepper, and cassava (e.g. Li et al. 2015; Svitashev et al. 2015; Li et al. 2016; Sauer et al. 2016; Hummel et al. 2018; Ortega et al. 2018).

In view of the above there is still a long felt and unmet need to confer non-transgenic herbicide-resistant traits to Cannabis. Especially to use technologies that enable precise modifications of DNA sequences for developing HR Cannabis which contributes to production cost savings, yield increase and more efficient control of weeds.

### SUMMARY OF THE INVENTION

It is therefore one object of the present invention to disclose a modified Cannabis plant exhibiting herbicide resistance (HR) as compared to a Cannabis plant absent of such modification, wherein said modified plant comprises at least one genetically modified HR-related gene comprising at least one mutation conferring herbicide resistance to the plant.

It is a further object of the present invention to disclose the modified Cannabis plant as defined above, wherein the at least one genetically modified HR-related gene comprises one or more base-editing driven nucleotide substitutions, resulting in a mutated HR-related gene conferring herbicide resistance to the plant.

It is a further object of the present invention to disclose the modified Cannabis plant as defined in any of the above, wherein the at least one mutation is in a HR-related gene encoding for a Cannabis HR-related protein selected from acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1).

It is a further object of the present invention to disclose the modified Cannabis plant as defined in any of the above, wherein the CsALS protein has an amino acid sequence as set forth in SEQ ID NO:3 or a functional variant thereof, and said protein is encoded by a genomic sequence as set forth in SEQ ID NO:1 or a functional variant thereof and/or by a coding sequence (CDS) as set forth in SEQ ID NO:2 or a functional variant thereof.

It is a further object of the present invention to disclose the modified Cannabis plant as defined in any of the above, wherein the CsCESA3 protein has an amino acid sequence as set forth in SEQ ID NO:6 or a functional variant thereof, and said protein is encoded by a genomic sequence as set forth in SEQ ID NO:4 or a functional variant thereof and/or by a coding sequence (CDS) as set forth in SEQ ID NO:5 or a functional variant thereof.

It is a further object of the present invention to disclose the modified Cannabis plant as defined in any of the above, wherein the CsEPSPS protein has an amino acid sequence as set forth in SEQ ID NO:9 or a functional variant thereof, and said protein is encoded by a genomic sequence as set forth in SEQ ID NO:7 or a functional variant thereof and/or by a coding sequence (CDS) as set forth in SEQ ID NO:8 or a functional variant thereof.

It is a further object of the present invention to disclose the modified Cannabis plant as defined in any of the above, wherein the CsSF3B1 protein has an amino acid sequence as set forth in SEQ ID NO:12 or a functional variant thereof, and said protein is encoded by a genomic sequence as set forth in SEQ ID NO:10 or a functional variant thereof and/or by a coding sequence (CDS) as set forth in SEQ ID NO:11 or a functional variant thereof.

It is a further object of the present invention to disclose the modified Cannabis plant as defined in any of the above, wherein said functional variant has at least 75% sequence identity to the corresponding nucleotide or amino acid sequence.

It is a further object of the present invention to disclose the modified Cannabis plant as defined in any of the above, wherein the mutated HR-related gene encodes a mutated CsALS protein comprising amino acid mutation at one or more positions selected from A118, P193, A201, W570 and S649 of SEQ ID NO: 3, said mutated protein confers herbicide resistance to a Cannabis plant.

It is a further object of the present invention to disclose the modified Cannabis plant as defined in any of the above, wherein the mutated HR-related gene encodes a mutated CsCESA3 protein comprising amino acid mutation at one or more positions selected from S998 and S1052 of SEQ ID NO: 6, said mutated protein confers herbicide resistance to a Cannabis plant.

It is a further object of the present invention to disclose the modified Cannabis plant as defined in any of the above, wherein the mutated HR-related gene encodes a mutated CsEPSPS protein comprising amino acid mutation at one or more positions selected from T181 and P185 of SEQ ID NO: 9, said mutated protein confers herbicide resistance to a Cannabis plant.

It is a further object of the present invention to disclose the modified Cannabis plant as defined in any of the above, wherein the mutated HR-related gene encodes a mutated CsSF3B1 protein comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant.

It is a further object of the present invention to disclose the modified Cannabis plant as defined in any of the above, wherein said plant comprises a base editing driven complex containing a nuclease-inactivated CRISPR/nuclease domain (Cas nickase or nCas) fused to a deaminase domain, and a gRNA comprising target sequence of the HR-related gene in the plant, said complex is driven by said gRNA to a target sequence in the herbicide resistance related gene in the plant so as to generate said one or more nucleotide substitutions.

It is a further object of the present invention to disclose the modified Cannabis plant as defined in any of the above, wherein said complex and said gRNA comprises at least one of the following: (a) fusion protein comprising said nuclease-inactivated CRISPR/nuclease domain fused to a deaminase, and said guide RNA; (b) an expression construct comprising a nucleotide sequence encoding said nuclease-inactivated CRISPR/nuclease domain fused to a deaminase, and said guide RNA; (c) a fusion protein comprising said nuclease-inactivated CRISPR/nuclease domain fused to a deaminase, and an expression construct comprising a nucleotide sequence encoding said guide RNA; (d) an expression construct comprising a nucleotide sequence encoding said nuclease-inactivated CRISPR/nuclease domain fused to a deaminase domain, and an expression construct comprising a nucleotide sequence encoding said guide RNA; and (e) an expression construct comprising a nucleotide sequence encoding said nuclease-inactivated CRISPR/nuclease domain fused to a deaminase domain and a nucleotide sequence encoding said guide RNA.

It is a further object of the present invention to disclose the modified Cannabis plant as defined in any of the above, wherein said base editing driven complex comprises at least one of: (a) a Cas9 nickase (nCas9) gene encoding an amino acid sequence as set forth in SEQ ID NO:14 fused to a deaminase and the target gene gRNA sequence, (b) a Ribonucleoprotein (RNP) complexes carrying nCas9 protein having an amino acid sequence as set forth in SEQ ID NO:14 fused to a deaminase protein and target gene gRNA sequence, or (c) RNP complex comprising the nCas9 protein having an amino acid sequence as set forth in SEQ ID NO:14 fused to a deaminase and the target gene gRNA sequence.

It is a further object of the present invention to disclose the modified Cannabis plant as defined in any of the above, wherein said mutation is introduced using mutagenesis, small interfering RNA (siRNA), microRNA (miRNA), artificial miRNA (amiRNA), DNA introgression, endonucleases or any combination thereof.

It is a further object of the present invention to disclose the modified Cannabis plant as defined in any of the above, wherein said mutation is introduced using CRISPR (Clustered Regularly Interspaced Short Palindromic Repeats) and CRISPR-associated (Cas) nuclease (CRISPR/Cas), Transcription activator-like effector nuclease (TALEN), Zinc Finger Nuclease (ZFN), meganuclease or any combination thereof.

It is a further object of the present invention to disclose the modified Cannabis plant as defined in any of the above, wherein said nuclease is selected from the group consisting of Cas3, Cas4, Cas5, Cas5e (orCasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmrl, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

It is a further object of the present invention to disclose the modified Cannabis plant as defined in any of the above, wherein the modified Cannabis plant comprises a mutated allele of at least one HR-related Cannabis gene encoding a protein selected from the group consisting of CsALS, CsEPSPS, CsCESA3, and CsSF3B1, said allele is a CRISPR/Cas-induced heritable mutated allele.

It is a further object of the present invention to disclose the modified Cannabis plant as defined in any of the above, wherein said plant is homozygous for said at least one mutated allele.

It is a further object of the present invention to disclose the modified Cannabis plant as defined in any of the above, wherein said at least one mutation is in the coding region of said gene, in the regulatory region of said gene, or an epigenetic factor.

It is a further object of the present invention to disclose the modified Cannabis plant as defined in any of the above, wherein said gRNA targets one or more of SEQ ID NOs: 1, 4, 7 and 10 or a portion thereof.

It is a further object of the present invention to disclose the modified Cannabis plant as defined in any of the above, wherein said gRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9) and TBN (Cas-phi).

It is a further object of the present invention to disclose the modified Cannabis plant as defined in any of the above, wherein said plant comprises at least one variant of a HR-related gene encoding a variant of a protein selected from the group consisting of CsALS, CsEPSPS, CsCESA3, and CsSF3B1 and any combination thereof, said protein variant is capable of conferring herbicide resistance to a Cannabis plant.

It is a further object of the present invention to disclose the modified Cannabis plant as defined in any of the above, wherein said protein variant is selected from the group consisting of: (a) CsALS variant comprising amino acid mutation at one of more positions selected from A118X, P193X, A201X, W570X and or S649X of SEQ ID NO: 3, said variant confers herbicide resistance to a Cannabis plant; (b) CsCESA3 variant comprising amino acid mutation at one of more positions selected from S998X and S1052X of SEQ ID NO: 6, said variant confers herbicide resistance to a Cannabis plant; (c) CsEPSPS variant comprising amino acid mutation at one of more positions selected from T181X and P185X of SEQ ID NO: 9, said variant confers herbicide resistance to a Cannabis plant; and (d) CsSF3B1 variant comprising amino acid mutation at one of more positions selected from SGR4 (K1033X/K1034X/G1035X), SGR5 (H1032X), and SGR6 (H1032X/A1049X) of SEQ ID NO: 12, said variant confers herbicide resistance to a Cannabis plant.

It is a further object of the present invention to disclose the modified Cannabis plant as defined in any of the above, wherein said plant is resistant to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof.

It is a further object of the present invention to disclose the modified Cannabis plant as defined in any of the above, wherein said Cannabis plant is selected from the group of species that includes, but is not limited to, Cannabis sativa (C. sativa), C. indica, C. ruderalis and any hybrid or cultivated variety of the genus Cannabis.

It is a further object of the present invention to disclose the modified Cannabis plant as defined in any of the above, wherein said plant is characterized by enhanced tolerance to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof, as compared to a Cannabis plant lacking said at least one modification.

It is a further object of the present invention to disclose a Cannabis plant, plant part or plant cell as defined in any of the above, wherein said plant is a transgene-free herbicide-resistant plant.

It is a further object of the present invention to disclose a plant part, plant cell or plant seed of a plant as defined in any of the above.

It is a further object of the present invention to disclose a tissue culture of regenerable cells, protoplasts or callus obtained from the modified Cannabis plant as defined in any of the above.

It is a further object of the present invention to disclose the modified Cannabis plant as defined in any of the above, wherein said plant genotype is obtainable by deposit under accession number with NCIMB Aberdeen AB21 9YA, Scotland, UK or with ATCC.

It is a further object of the present invention to disclose an isolated nucleic acid comprising a genomic nucleotide sequence having at least 75% sequence similarity to a genomic nucleotide sequence selected from the group consisting of SEQ ID NO:1, SEQ ID NO:4, SEQ ID NO:7 and SEQ ID NO:10.

It is a further object of the present invention to disclose an isolated nucleic acid variant of a genomic nucleotide sequence selected from the group consisting of SEQ ID NO:1, SEQ ID NO:4 and SEQ ID NO:7, SEQ ID NO:10, said variant is capable of conferring herbicide resistance to a Cannabis plant.

It is a further object of the present invention to disclose the isolated nucleic acid variant as defined in any of the above, wherein the genomic nucleotide-sequence variant is selected from the group consisting of: (a) variant of SEQ ID NO:1 encoding a mutated protein comprising amino acid mutation at one of more positions selected from A118X, P193X, A201X, W570X and or S649X of SEQ ID NO: 3, said variant confers herbicide resistance to a Cannabis plant; (b) variant of SEQ ID NO:4 encoding a mutated protein comprising amino acid mutation at one of more positions selected from S998X and S1052X of SEQ ID NO: 6, said variant confers herbicide resistance to a Cannabis plant; (c) variant of SEQ ID NO:7 encoding a mutated protein comprising amino acid mutation at one of more positions selected from T181X and P185X of SEQ ID NO: 9, said variant confers herbicide resistance to a Cannabis plant; and (d) variant of SEQ ID NO:10 encoding a mutated protein comprising amino acid mutation at one of more positions selected from SGR4 (K1033X/K1034X/G1035X), SGR5 (H1032X), and SGR6 (H1032X/A1049X) of SEQ ID NO: 12, said variant confers herbicide resistance to a Cannabis plant.

It is a further object of the present invention to disclose an isolated nucleic acid comprising a coding nucleotide sequence (CDS) having at least 75% sequence similarity to a CDS selected from the group consisting of SEQ ID NO:2, SEQ ID NO:5, SEQ ID NO:8 and SEQ ID NO:11.

It is a further object of the present invention to disclose an isolated nucleic acid variant of a coding nucleotide sequence (CDS) having a CDS selected from the group consisting of SEQ ID NO:2, SEQ ID NO:5, SEQ ID NO:8 and SEQ ID NO:11, said variant is capable of conferring herbicide resistance to a Cannabis plant.

It is a further object of the present invention to disclose the isolated nucleic acid variant as defined in any of the above, wherein the CDS variant is selected from the group consisting of: (a) variant of SEQ ID NO:2 encoding a mutated protein comprising amino acid mutation at one of more positions selected from A118X, P193X, A201X, W570X and or S649X of SEQ ID NO: 3, said variant confers herbicide resistance to a Cannabis plant; (b) variant of SEQ ID NO:5 encoding a mutated protein comprising amino acid mutation at one of more positions selected from S998X and S1052X of SEQ ID NO: 6, said variant confers herbicide resistance to a Cannabis plant; (c) variant of SEQ ID NO:8 encoding a mutated protein comprising amino acid mutation at one of more positions selected from T181X and P185X of SEQ ID NO: 9, said variant confers herbicide resistance to a Cannabis plant; and (d) variant of SEQ ID NO:11 encoding a mutated protein comprising amino acid mutation at one of more positions selected from SGR4 (K1033X/K1034X/G1035X), SGR5 (H1032X), and SGR6 (H1032X/A1049X) of SEQ ID NO: 12, said variant confers herbicide resistance to a Cannabis plant.

It is a further object of the present invention to disclose an isolated amino acid comprising an amino acid sequence having at least 75% sequence similarity to an amino acid sequence selected from the group consisting of SEQ ID NO:3, SEQ ID NO:6, SEQ ID NO:9 and SEQ ID NO:12.

It is a further object of the present invention to disclose an isolated protein variant of an amino acid sequence selected from the group consisting of SEQ ID NO:3, SEQ ID NO:6, SEQ ID NO:9 and SEQ ID NO:12, said variant is capable of conferring herbicide resistance to a Cannabis plant.

It is a further object of the present invention to disclose the isolated protein variant as defined in any of the above, wherein said protein variant is selected from the group consisting of: (a) variant of SEQ ID NO:3 comprising amino acid mutation at one of more positions selected from A118X, P193X, A201X, W570X and or S649X, said variant confers herbicide resistance to a Cannabis plant; (b) variant of SEQ ID NO:6 comprising amino acid mutation at one of more positions selected from S998X and S1052X, said variant confers herbicide resistance to a Cannabis plant; (c) variant of SEQ ID NO:9 comprising amino acid mutation at one of more positions selected from T181X and P185X, said variant confers herbicide resistance to a Cannabis plant; and (d) variant of SEQ ID NO:12 comprising amino acid mutation at one of more positions selected from SGR4 (K1033X/K1034X/G1035X), SGR5 (H1032X), and SGR6 (H1032X/A1049X), said variant confers herbicide resistance to a Cannabis plant.

It is a further object of the present invention to disclose a CsALS variant, compared with wild-type CsALS, said CsALS variant comprises amino acid mutation at one of more positions selected from A118X, P193X, A201X, W570X and or S649X in SEQ ID NO: 3, said variant confers herbicide resistance to a Cannabis plant.

It is a further object of the present invention to disclose a CsCESA3 variant, compared with wild-type CsCESA3, said CsCESA3 variant comprises amino acid mutation at one of more positions selected from S998X and/or S1052X in SEQ ID NO: 6, said variant confers herbicide resistance to a Cannabis plant.

It is a further object of the present invention to disclose a CsEPSPS variant, compared with wild-type CsEPSPS, said CsEPSPS variant comprises amino acid mutation at one of more positions selected from T181X and/or P185X in SEQ ID NO: 9, said variant confers herbicide resistance to a Cannabis plant.

It is a further object of the present invention to disclose a CsSF3B1 variant, compared with wild-type CsSF3B1, said CsSF3B1 variant comprises amino acid mutation at one of more positions selected from SGR4 (K1033X and/orK1034X and/or G1035X), SGR5 (H1032X), and SGR6 (H1032X and/orA1049X) in SEQ ID NO: 12, said variant confers herbicide resistance to a Cannabis plant.

It is a further object of the present invention to disclose an isolated nucleic acid comprising a nucleotide sequence encoding a variant as defined in any of the above.

It is a further object of the present invention to disclose a Cannabis plant comprising the variant as defined in any of the above and/or the nucleic acid as defined in any of the above.

It is a further object of the present invention to disclose an expression cassette and/or construct comprising a nucleotide sequence encoding a variant as defined in any of the above operably linked to a regulatory sequence.

It is a further object of the present invention to disclose use of the variant as defined in any of the above, the isolated nucleic acid as defined in any of the above, and/or the expression cassette and/or construct as defined in any of the above, in the generation of herbicide-resistant Cannabis plants.

It is a further object of the present invention to disclose a method of producing a herbicide-resistant Cannabis plant, comprising introducing the isolated nucleic acid variant as defined in any of the above, and/or the expression cassette and/or construct as defined in any of the above into a Cannabis plant.

It is a further object of the present invention to disclose a method of producing a herbicide-resistant (HR) Cannabis plant as defined in any of the above, said method comprises steps of genetically modifying at least one HR-related gene by introducing at least one mutation within said at least one HR-related gene so as to confer herbicide resistance to the plant.

It is a further object of the present invention to disclose the method as defined in any of the above, comprising steps of introducing by base-editing, a modification in a HR-related gene in the plant genome, so as to generate one or more nucleotide substitutions in the HR-related gene, wherein said one or more nucleotide substitutions confers herbicide resistance to the Cannabis plant, optionally the method further comprises a step of screening the plant for herbicide resistance.

It is a further object of the present invention to disclose the method as defined in any of the above, wherein said steps of introducing by base-editing a modification in a HR-related gene in the plant genome, comprising introducing into the plant a complex containing a nuclease-inactivated CRISPR/nuclease domain fused to a deaminase domain, and a gRNA comprising target sequence of the HR-related gene in the plant, said gRNA drive said complex to the target sequence in the herbicide resistance related gene in the plant so as to generate said one or more nucleotide substitutions.

It is a further object of the present invention to disclose the method as defined in any of the above wherein said complex and said gRNA comprises at least one of the following: (a) a fusion protein comprising said nuclease-inactivated CRISPR/nuclease domain fused to a deaminase, and said guide RNA; (b) an expression construct comprising a nucleotide sequence encoding said nuclease-inactivated CRISPR/nuclease domain fused to a deaminase, and said guide RNA; (c) a fusion protein comprising said nuclease-inactivated CRISPR/nuclease domain fused to a deaminase, and an expression construct comprising a nucleotide sequence encoding said guide RNA; (d) an expression construct comprising a nucleotide sequence encoding said nuclease-inactivated CRISPR/nuclease domain fused to a deaminase domain, and an expression construct comprising a nucleotide sequence encoding said guide RNA; and (e) an expression construct comprising a nucleotide sequence encoding said nuclease-inactivated CRISPR/nuclease domain fused to a deaminase domain and a nucleotide sequence encoding said guide RNA.

It is a further object of the present invention to disclose the method as defined in any of the above, wherein said base editing driven complex comprises at least one of: (a) a Cas9 nickase (nCas9) gene encoding an amino acid sequence as set forth in SEQ ID NO:14 fused to a deaminase and the target gene gRNA sequence, (b) a Ribonucleoprotein (RNP) complexes carrying nCas9 protein having an amino acid sequence as set forth in SEQ ID NO:14 fused to a deaminase protein and target gene gRNA sequence, or (c) RNP complex comprising the nCas9 protein having an amino acid sequence as set forth in SEQ ID NO:14 fused to a deaminase and the target gene gRNA sequence.

It is a further object of the present invention to disclose the method as defined in any of the above, wherein the at least one mutation is introduced into a HR-related gene encoding for a Cannabis HR-related protein selected from acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1).

It is a further object of the present invention to disclose the method as defined in any of the above, wherein said CsALS protein has an amino acid sequence as set forth in SEQ ID NO:3 or a functional variant thereof, and said protein is encoded by a genomic sequence as set forth in SEQ ID NO:1 or a functional variant thereof and/or by a coding sequence (CDS) as set forth in SEQ ID NO:2 or a functional variant thereof.

It is a further object of the present invention to disclose the method as defined in any of the above, wherein said CsCESA3 protein has an amino acid sequence as set forth in SEQ ID NO:6 or a functional variant thereof, and said protein is encoded by a genomic sequence as set forth in SEQ ID NO:4 or a functional variant thereof and/or by a coding sequence (CDS) as set forth in SEQ ID NO:5 or a functional variant thereof.

It is a further object of the present invention to disclose the method as defined in any of the above, wherein said CsEPSPS protein has an amino acid sequence as set forth in SEQ ID NO:9 or a functional variant thereof, and said protein is encoded by a genomic sequence as set forth in SEQ ID NO:7 or a functional variant thereof and/or by a coding sequence (CDS) as set forth in SEQ ID NO:8 or a functional variant thereof.

It is a further object of the present invention to disclose the method as defined in any of the above, wherein said CsSF3B1 protein has an amino acid sequence as set forth in SEQ ID NO:12 or a functional variant thereof, and said protein is encoded by a genomic sequence as set forth in SEQ ID NO:10 or a functional variant thereof and/or by a coding sequence (CDS) as set forth in SEQ ID NO:11 or a functional variant thereof.

It is a further object of the present invention to disclose the method as defined in any of the above, wherein said functional variant has at least 75% sequence identity to the corresponding nucleotide or amino acid sequence.

It is a further object of the present invention to disclose the method as defined in any of the above, wherein the mutated HR-related gene encodes a mutated CsALS protein, comprising amino acid mutation at one or more positions selected from A118, P193, A201, W570 and S649 of SEQ ID NO: 3, said mutated protein confers herbicide resistance to a Cannabis plant.

It is a further object of the present invention to disclose the method as defined in any of the above, wherein the mutated HR-related gene encodes a mutated CsCESA3 protein, comprising amino acid mutation at one or more positions selected from S998 and S1052 of SEQ ID NO: 6, said mutated protein confers herbicide resistance to a Cannabis plant.

It is a further object of the present invention to disclose the method as defined in any of the above, wherein the mutated HR-related gene encodes a mutated CsEPSPS protein, comprising amino acid mutation at one or more positions selected from T181 and P185 of SEQ ID NO: 9, said mutated protein confers herbicide resistance to a Cannabis plant.

It is a further object of the present invention to disclose the method as defined in any of the above, wherein the mutated HR-related gene encodes a mutated CsSF3B1 protein, comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant.

It is a further object of the present invention to disclose the method as defined in any of the above, wherein said gRNA targets one or more of SEQ ID NOs: 1, 4, 7 and 10 or a portion thereof.

It is a further object of the present invention to disclose the method as defined in any of the above, wherein said plant is resistant to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof.

It is a further object of the present invention to disclose a method of identifying a variant or mutant of a Cannabis herbicide-resistance (HR) related protein, wherein said variant or mutant is capable of conferring herbicide resistance to a Cannabis plant, said method comprising: (a) generating a HR Cannabis plant by the method as defined in any of the above; and (b) determining the sequence of the HR-related gene and/or the encoded HR-related protein in the resulting herbicide resistant plant, thereby identifying the sequence of the variant or mutant.

It is a further object of the present invention to disclose the method as defined in any of the above, wherein said HR-related protein is selected from the group consisting of CsALS, CsEPSPS, CsCESA3, and CsSF3B1.

It is a further object of the present invention to disclose the method as defined in any of the above, wherein said HR-related protein variant or mutant has at least one mutation in an amino acid sequence having at least 75% similarity to an amino acid sequence selected from the group consisting of SEQ ID NO:3, SEQ ID NO:6, SEQ ID NO:9 and SEQ ID NO:12.

It is a further object of the present invention to disclose the method as defined in any of the above, wherein said HR-related protein variant or mutant is selected from the group consisting of: (a) variant of SEQ ID NO:3 comprising amino acid mutation at one of more positions selected from A118X, P193X, A201X, W570X and or S649X, said variant confers herbicide resistance to a Cannabis plant; (b) variant of SEQ ID NO:6 comprising amino acid mutation at one of more positions selected from S998X and S1052X, said variant confers herbicide resistance to a Cannabis plant; (b) variant of SEQ ID NO:9 comprising amino acid mutation at one of more positions selected from T181X and P185X, said variant confers herbicide resistance to a Cannabis plant; and (c) variant of SEQ ID NO:12 comprising amino acid mutation at one of more positions selected from SGR4 (K1033X/K1034X/G1035X), SGR5 (H1032X), and SGR6 (H1032X/A1049X), said variant confers herbicide resistance to a Cannabis plant.

It is a further object of the present invention to disclose a variant or mutant of a herbicide resistance-related protein, which is identified by the method as defined in any of the above, wherein said variant is capable of conferring herbicide resistance to a plant.

It is a further object of the present invention to disclose an isolated nucleic acid comprising a nucleotide sequence encoding the variant or mutant as defined in any of the above.

It is a further object of the present invention to disclose an expression cassette and/or construct comprising a nucleotide sequence encoding the variant or mutant as defined in any of the above, operably linked to a regulatory sequence.

It is a further object of the present invention to disclose a use of the variant or mutant as defined in any of the above, the isolated nucleic acid as defined in any of the above, and/or the expression cassette and/or construct as defined in any of the above, in the generation of Cannabis herbicide-resistant plants.

It is a further object of the present invention to disclose a method of producing a herbicide-resistant Cannabis plant, comprising introducing the isolated nucleic acid as defined in any of the above, and/or the expression cassette and/or construct as defined in any of the above, into a Cannabis plant.

It is a further object of the present invention to disclose a herbicide-resistant Cannabis plant, obtainable by the method as defined in any of the above.

It is a further object of the present invention to disclose a transgenic Cannabis plant or seed, comprising the genetically modified cell as defined in any of the above.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

Weeds are one of the major threats to reduce agricultural productivity, as competing with crops for light, water, space, and nutritional resources. As a result, herbicides are widely used in agronomic crops as the primary method of weed control. Accordingly, extensive efforts to develop crop varieties with herbicide tolerance or resistance have been made to provide cost effective tools to manage weeds.

Due to the rapid appearance of the herbicide-tolerant weeds combined with a lack of novel herbicides, there is a need for new methods that confer herbicide resistance to crops.

The present invention provides a modified Cannabis plant exhibiting herbicide resistance (HR) as compared to a Cannabis plant absent of such modification. The modified plant comprises at least one mutated HR-related gene conferring herbicide resistance to the plant.

According to one embodiment, the present invention provides a modified Cannabis plant exhibiting herbicide resistance (HR) as compared to a Cannabis plant absent of such modification, wherein the d modified plant comprises at least one genetically modified HR-related gene comprising at least one mutation conferring herbicide resistance to the plant.

According to a further embodiment, the present invention provides a modified Cannabis plant exhibiting herbicide resistance (HR) as compared to a Cannabis plant absent of such modification, wherein said plant comprises at least one base-editing driven modification in a HR-related gene in the plant genome, resulting in one or more nucleotide substitutions in the HR-related gene conferring herbicide resistance to the plant. The present invention further provides methods for producing the aforementioned modified Cannabis plant using genome editing or other genome modification techniques.

Introducing point mutations that confer resistance to herbicides is not easy to achieve by traditional breeding, and the GM technology cannot confer non-transgenic herbicide-resistant traits to crops. Thus, plant genome editing technologies that enable precise modifications of DNA sequences for loss of-function and gain-of-function mutations might offer a solution for crop improvement.

The present invention describes, for the first time, the development of herbicide-resistant (HR) Cannabis plants by the CRISPR/Cas-mediated gene editing technology, targeting on Cannabis endogenous genes such as acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1).

According to aspects of the present invention, the CRISPR system with CRISPR-associated protein 9 (Cas9) is a useful method for plant genome editing, in which Cas9 is directed by a single guide RNA (sgRNA) to a specific DNA sequence and generates site-specific double-strand breaks (DSBs). The resulting DSBs can be repaired by error-prone non-homologous end joining (NHEJ), often producing nucleotide insertions/deletions (indels) and/or substitutions, and also by homology-directed repair (HDR) when homologous donor templates are present at the time of DSB formation.

The present invention employs herbicide-resistant plants generated by CRISPR/Cas-mediated gene editing to accomplish programmable base-editing. This is achieved by fusing deaminase enzyme (e.g. adenine deaminase or cytidine deaminase) to a catalytically impaired CRISPR/Cas mutant (i.e. Cas9 nickase or nCas9, see mutated D10A, H840A Cas9 of SEQ ID NO:14), leading to A-to-G or C-to-T substitution without the introduction of a double-strand break (DSB) in the DNA. Without wishing to be bound by theory, these base editors drive the nCas9-gRNA complex to the target locus and enable deamination on the non-complementary strand.

Thus, according to an embodiment of the present invention, the base editors are herein applied as a precise and effective method to develop HR Cannabis plants.

It is further within the scope of the present invention that for the development of HR Cannabis plants by the CRISPR/nuclease system, it is important to select target genes for the gene editing. According to one embodiment of the present invention, Cannabis endogenous genes selected for targeting by gene editing include acetolactate synthase (ALS), 5-enolpyruvylshikimate-3-phosphate synthase (EPSPS), cellulose synthase A catalytic subunit 3 (CESA3), and splicing factor 3B subunit 1 (SF3B1).

According to further embodiments, the present invention provides DNA genomic sequence, coding sequence (CDS) and amino acid sequence for each of the aforementioned endogenous Cannabis genes (see SEQ ID NO:1-12). These Cannabis-originated genes are candidates to confer resistance to herbicides by CRISPR/Cas-mediated gene editing.

Without wishing to be bound by theory, glyphosate (N-phosphonomethyl-glycine) is a non-selective and broad-spectrum systemic herbicide that competes with the substrate PEP at the binding site and inhibits the shikimate pathway in the chloroplast of plants.

The EPSPS is 5-enolpyruvylshikimate-3-phosphate (EPSP) synthase that catalyzes the chemical reaction where the two substrates are phosphoenolpyruvate (PEP) and 3-phospho-shikimate, and its two products are inorganic phosphate and 5-enolpyruvylshikimate-3-phosphate (EPSP).

It is acknowledged that this enzyme is essential for the synthesis of aromatic compounds including essential aromatic amino acids in most organisms. It is also notable that this enzyme is absent in mammals, which makes EPSPS as an attractive target for the development of herbicides, such as glyphosate. A glyphosate-resistant version of this gene has been used in genetically modified crops.

The enzyme acetolactate synthase (ALS) also known as acetohydroxyacid synthase (AHAS) is found in plants and microorganisms, but not in animals. This enzyme catalyzes the first step in the biosynthesis of the essential branched-chain amino acids.

It is herein acknowledged that five of the most widely used commercial herbicides exert their activity by inhibiting ALS, which include sulfonylurea (SU), imidazolinone (IMI), triazolopyrimidine, pyrimidinyl-benzoate and sulfonyl-aminocabonyl-triazolinone. Accordingly, Cannabis endogenous ALS genome-edited mutants resistant to these herbicides are desirable and aimed by the present invention.

Reference is now made to Cellulose synthase catalytic subunits (CESAs), which catalyzes the conversion of UDPglucose to cellulose. CBI compounds are known to inhibit CESAs. CBI is herein referred to as a cellulose biosynthesis-inhibiting chemical compound which is a potential herbicide that efficiently inhibits the growth of various weeds and dicotyledonous crops. Cannabis plants carrying targeted CRISPR/Cas-mediated gene editing mutation(s) at the endogenous CsCESA3 gene to confer resistant to C17 is another object within the scope of the current invention.

Reference is now made to splicing factor 3B subunit 1 (SF3B1) gene as a target for resistance to splicing inhibitors. In plants, naturally occurring splicing inhibitors, such as pladienolide B and herboxidiene produced by Streptomyces sp., have significant effects on transcriptome-wide splicing repression, which can be applied to plants as herbicides.

It is within the scope of the current invention to achieve, using a CRISPR/Cas-based directed platform, genome editing of Cannabis SF3B1 (Cs SF3B1) endogenous gene, resulting in resistance to herboxidiene, also referred to as SF3B1-GEX1A-Resistant or SGR.

According to one embodiment of the present invention, the wild-type CsALS protein has an amino acid sequence as set forth in SEQ ID NO:3 or a functional variant thereof, and said protein is encoded by a genomic sequence as set forth in SEQ ID NO:1 or a functional variant thereof and/or by a coding sequence (CDS) as set forth in SEQ ID NO:2 or a functional variant thereof.

According to a further embodiment of the present invention, the wild-type CsCESA3 protein has an amino acid sequence as set forth in SEQ ID NO:6 or a functional variant thereof, and said protein is encoded by a genomic sequence as set forth in SEQ ID NO:4 or a functional variant thereof and/or by a coding sequence (CDS) as set forth in SEQ ID NO:5 or a functional variant thereof.

According to a further embodiment of the present invention, the wild-type CsEPSPS protein has an amino acid sequence as set forth in SEQ ID NO:9 or a functional variant thereof, and said protein is encoded by a genomic sequence as set forth in SEQ ID NO:7 or a functional variant thereof and/or by a coding sequence (CDS) as set forth in SEQ ID NO:8 or a functional variant thereof.

According to a further embodiment of the present invention, the wild-type CsSF3B1 protein has an amino acid sequence as set forth in SEQ ID NO:12 or a functional variant thereof, and said protein is encoded by a genomic sequence as set forth in SEQ ID NO:10 or a functional variant thereof and/or by a coding sequence (CDS) as set forth in SEQ ID NO:11 or a functional variant thereof. For the application of genome editing, the above HR-related genes are herein targeted to develop HR Cannabis. For this end, it is needed to identify amino acid change(s) that are necessary for the generation of enzymes insensitive to the corresponding herbicides. It is important that according to main aspects of the invention, the targeted amino acid changes should confer improved herbicide tolerance without causing loss of the enzymatic catalytic activity.

According to some aspects of the present invention, using the natural site-mutants of genes known for herbicide resistance, the editing of the endogenous genes by various CRISPR/Cas-based technologies, is herein performed in Cannabis. For the gene editing of CsEPSPS, base editors have been used to generate similar or corresponding mutations to the natural T1021/P106S mutant (2mEPSPS) from Z. mays, i.e., a double mutant with a threonine to isoleucine mutation and a proline to serine or alanine mutation.

For the gene editing of CsALS, base editors have been used to generate substitution mutations on proline, tryptophan, serine, alanine, and glycine residues (A122, P197, W574, S653, and G654 in Arabidopsis ALS) or corresponding residues that have been known to commonly encounter as natural mutations.

It is further within the scope that CBI compounds are known to inhibit cellulose synthase catalytic subunits (CESAs) that catalyze the conversion of UDPglucose to cellulose. It is acknowledged that a mutagenesis suppressor screen has identified C17-tolerant mutants carrying single nucleotide missense mutations at CESAs, for example, S983F in CESA3 (Hu et al. 2016 incorporated herein by reference). Corresponding targeted mutations through CRISPR/Cas-mediated gene editing is performed on CsCESA3 endogenous gene to develop HR Cannabis plants.

According to a further embodiment of the present invention, the Cannabis splicing factor 3B subunit 1 (CsSF3B1) gene is a target for resistance to splicing inhibiting herbicides. Using CRISPR/Cas9-based directed platform, SF3B1 mutants resistant to herboxidiene (SGR) are obtained in amino acid sites corresponding to SGR4 (K1049R/K1050E/G1051H), SGR5 (H1048Q/K1049 deletion), and SGR6 (H1048Q/K1049 deletion/A1064S) (Butt et al. 2019; incorporated herein by reference).

Thus, it is within the scope of the present invention, that using targeted base-editing, CsALS mutants resistant to herbicides are obtained in amino acid sites corresponding to or functionally equivalent to at least one of W548L, S627I, A96V, P190S, P174S/F and G631X (X represents base editing on G631 with 11 different amino acid substitutions).

It is further within the scope of the present invention, that using targeted base-editing, CsEPSPS mutants resistant to herbicides are obtained in amino acid sites corresponding to or functionally equivalent to at least one of T1731/P177S, T1781/P182A, T1791/P183S and T1021/P106A.

It is further within the scope of the present invention, that using targeted base-editing, CsCESA3 mutants resistant to herbicides are obtained in amino acid sites corresponding to or functionally equivalent to S983F.

It is further within the scope of the present invention, that using targeted base-editing, CsSF3B1 mutants resistant to herbicides are obtained in amino acid sites corresponding to or functionally equivalent to at least one of SF3B1-GEX1A-Resistant (SGR) mutants selected from SGR4 (K1049R/K1050E/G1051H), SGR5 (H1048Q/K1049 deletion) and SGR6 (H1048Q/K1049 deletion/A1064S).

In further embodiments of the present invention, targeted base-editing is performed in at least one of the Cannabis gene sequences selected from CsALS as set forth in SEQ ID NO:1 or SEQ ID NO:2, encoding a protein comprising amino acid sequence as set forth in SEQ ID NO:3; CsCESA3 as set forth in SEQ ID NO:4 or SEQ ID NO:5, encoding a protein comprising amino acid sequence as set forth in SEQ ID NO:6; CsEPSPS as set forth in SEQ ID NO:7 or SEQ ID NO:8, encoding a protein comprising amino acid sequence as set forth in SEQ ID NO:9; and CsSF3B1 as set forth in SEQ ID NO:10 or SEQ ID NO:11, encoding a protein comprising amino acid sequence as set forth in SEQ ID NO:12, to achieve herbicide resistance (HR) in Cannabis.

According to one embodiment of the present invention, the plant comprises a mutated CsALS protein, compared with wild-type CsALS, comprising amino acid mutation at one or more positions selected from A118, P193, A201, W570 and S649 of SEQ ID NO: 3, said mutated protein confers herbicide resistance to a Cannabis plant.

According to a further embodiment of the present invention, the plant comprises a mutated CsCESA3 protein, compared with wild-type CsCESA3, comprising amino acid mutation at one or more positions selected from S998 and S1052 of SEQ ID NO: 6, said mutated protein confers herbicide resistance to a Cannabis plant.

According to a further embodiment of the present invention, the plant comprises a mutated CsEPSPS protein, compared with wild-type CsEPSPS, comprising amino acid mutation at one or more positions selected from T181 and P185 of SEQ ID NO: 9, said mutated protein confers herbicide resistance to a Cannabis plant.

According to a further embodiment of the present invention, the plant comprises a mutated CsSF3B1 protein, compared with wild-type CsSF3B1, comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant. According to some aspects of the present invention, CRISPR/Cas-mediated gene editing in CsALS is targeted at generating Cannabis resistance to herbicides including Chlorsulfuron, Bispyribac sodium, Imazamox, Tribenuron, Nicosulfuron, Mesosulfuron and Imazapic.

According to some further aspects of the present invention, CRISPR/Cas9-mediated gene editing in CsCESA3 is targeted at generating Cannabis resistance to herbicides including C17 (Cellulose biosynthesis-inhibiting chemical compound).

According to some further aspects of the present invention, CRISPR/Cas-mediated gene editing in CsEPSPS is performed to generate Cannabis resistance to herbicides including Glyphosate.

According to some further aspects of the present invention, CRISPR/Cas-mediated gene editing in CsSF3B1 is performed to generate Cannabis resistance to herbicides including Herboxidiene (GEX1A).

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Cannabis species or strain or variety or to sibling plant, or one or more individuals having one or both parents in common.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

It is further within the scope that the term “plant” includes a whole plant and any descendant, cell, tissue, or part of a plant. The term “plant parts” include any part (s) of a plant, including, for example and without limitation: seed; a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. It is noted that some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

According to further aspects of the present invention, plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

The term “plant cell” used herein refers to a structural and physiological unit of a plant, comprising a protoplast and a cell wall. The plant cell may be in a form of an isolated single cell or an aggregate of cells (e.g., a friable callus and a cultured cell), or as a part of higher organized unit such as, for example, plant tissue, a plant organ, or a whole plant. Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “protoplast” as used herein, refers to a plant cell that had its cell wall completely or partially removed, with the lipid bilayer membrane thereof naked, and thus includes protoplasts, which have their cell wall entirely removed, and spheroplasts, which have their cell wall only partially removed, but is not limited thereto. Typically, a protoplast is an isolated plant cell without cell walls which has the potency for regeneration into cell culture or a whole plant.

As used herein, the term “progeny” or “progenies” refers in a non-limiting manner to any subsequent generation of the plant, including offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. mutation in at least one herbicide resistance-related gene encoding CsALS, CsCESA3, CsEPSPS and/or CsSF3B1 and conferring herbicide resistance to the plant.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

The term “herbicide resistance” or “HR” as used herein encompass the definition according to the Weed Science Society of America (WSSA) where herbicide resistance is defined as the inherited ability of a plant to survive and reproduce following exposure to a dose of herbicide normally lethal to the wild type.

According to other aspects of the present invention, herbicide tolerance is defined as the inherent ability of a species to survive and reproduce after herbicide treatment. It may be understood that in this aspect, there was no selection or genetic manipulation to make the plant tolerant to herbicides. Based on these definitions, the current invention relates to herbicide-resistant (HR) Cannabis plants (rather than herbicide-tolerant plants) that can be developed by the genome editing technology.

The term “genome” as applies to plant cells, encompasses chromosomal DNA found within the nucleus, and organelle DNA found within subcellular components (e.g., mitochondrial, plastid) of the cell.

A “genetically modified plant” includes, in the context of the present invention, a plant which comprises within its genome an exogenous polynucleotide. For example, the exogenous polynucleotide is stably integrated within the genome such that the polynucleotide is passed on to successive generations. The exogenous polynucleotide may be integrated into the genome alone or as part of a recombinant DNA construct. The modified gene or expression regulatory sequence means that, in the plant genome, comprises one or more nucleotide substitution, deletion, or addition. For example, a genetically modified plant obtained by the present invention may contain one or more C to T substitutions relative to the wild type plant (corresponding plant that is not genetically modified).

As used herein, the term “exogenous” with respect to sequence, means a sequence that originates from a foreign species, or, if from the same species, is substantially modified from its native form in composition and/or genomic locus by deliberate human intervention.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with improved domestication traits are generated using genome editing mechanism. This technique enables to achieve in planta modification of specific genes that relate to and/or control the flowering time and plant architecture in the Cannabis plant.

The term “genome editing”, or “gene editing”, or “genome/genetic modification”, or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

### Genome Editing Glossary

Cas=CRISPR-associated genes Indel=insertion and/or deletion
Cas9, Csn1=a CRISPR-associated protein NHEJ=Non-Homologous End Joining containing two nuclease domains, that is PAM=Protospacer-Adjacent Motif programmed by small RNAs to cleave DNA RuvC=an endonuclease domain named for
crRNA=CRISPR RNA an E. coli protein involved in DNA repair
dCAS9=nuclease-deficient Cas9 sgRNA=single guide RNA
DSB=Double-Stranded Break tracrRNA, trRNA=trans-activating crRNA
gRNA=guide RNA TALEN=Transcription-Activator Like
HDR=Homology-Directed Repair Effector Nuclease
HNH=an endonuclease domain named ZFN=Zinc-Finger Nuclease for characteristic histidine and asparagine residues

According to specific aspects of the present invention, the CRISPR (Clustered Regularly Interspaced Short Palindromic Repeats) and CRISPR-associated (Cas) genes are used for the first time for generating genome modification in targeted genes in the Cannabis plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

The terms “Cas9 nuclease” and “Cas9” or CRISPR/Cas can be used interchangeably herein, and refer to a RNA directed nuclease, including the Cas9 protein or fragments thereof (such as a protein comprising an active DNA cleavage domain of Cas9 and/or a gRNA binding domain of Cas9). Cas9 is a component of the CRISPR/Cas (clustered regularly interspaced short palindromic repeats and its associated system) genome editing system, which targets and cleaves a DNA target sequence to form a DNA double strand breaks (DSB) under the guidance of a guide RNA.

According to further aspects of the invention, Cas protein, such as Cas9 (also known as Csn1) participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand to gRNA, while the RuvC domain cleaves the non-complementary strand.

It is further noted that the double-stranded endonuclease activity of Cas9 also requires that a short conserved sequence, (2-5 nts) known as protospacer-associated motif (PAM), follows immediately 3′—of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9 or nCas9).

A general exemplified CRISPR/Cas9 mechanism of action is depicted by Xie, Kabin, and Yinong Yang. “RNA-guided genome editing in plants using a CRISPR-Cas system.” Molecular plant 6.6 (2013): 1975-1983. As shown in this publication, which is incorporated herein by reference, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

As the DNA-cutting such as CRISPR-Cas9 and related genome-editing tools mainly originate from bacteria, Cas proteins apparently evolving in viruses that infect bacteria, are also within the scope of the present invention. For example, the most compact Cas variants were found in bacteriophages (bacteria-infecting viruses) and they herein referred to as CasΦ (Cas-phi).

It is therefore within the scope of the present invention that the nuclease used for base-editing of a predetermined Cannabis HR-related gene may be selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmrl, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

The term “base editing” or “base-editing” in the context of the present invention refers to a genome editing approach that uses components from CRISPR systems together with other enzymes to directly introduce point mutations into cellular DNA or RNA without making double-stranded DNA breaks (DSBs). It is within the scope that DNA base editors comprise a catalytically disabled or inactivated nuclease fused to a nucleobase deaminase enzyme or a DNA glycosylase inhibitor. It is acknowledged that RNA base editors achieve analogous changes using components that target RNA. According to aspects of the present invention, base editors directly convert one base or base pair into another, enabling the efficient introduction of specific and precise point mutations in non-dividing cells without generating excess undesired editing byproducts such as indels, translocations, and rearrangements derived from DSBs created by nucleases such as Cas9.

It is further within the scope of the current invention that DNA base editors (BEs) comprise fusions between a catalytically impaired Cas nuclease and a base-modification enzyme that operates on single-stranded DNA (ssDNA) but not double-stranded DNA (dsDNA). Without wishing to be bound by theory, it is noted that upon binding to its target locus in DNA, base pairing between the guide RNA and target DNA strand leads to displacement of a small segment of single-stranded DNA in an “R-loop”. DNA bases within this single-stranded DNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template.

It is within the scope of the present invention that two classes of DNA base editor have been described: cytosine base editors (CBEs) which convert a C⋅G base pair into a T⋅A base pair, and adenine base editors (ABEs) which convert an A⋅T base pair to a G⋅C base pair. Together, CBEs and ABEs can mediate all four possible transition mutations (C to T, A to G, T to C, and G to A). In RNA, targeted adenosine conversion to inosine has been used in both antisense and Cas13-guided RNA-targeting methods. In the current invention, base editing techniques are used to generate targeted mutations in HR-related genes (including genes encoding CsALS, CsCESA3, CsEPSPS and CsSF3B1) in Cannabis to confer herbicide resistance to the plant.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “guide RNA” or “gRNA” can be used interchangeably herein, and are composed of crRNA and tracrRNA molecules forming complexes through partial complement, wherein crRNA comprises a sequence that is sufficiently complementary to a target sequence for hybridization and directs the CRISPR complex (Cas9+crRNA+tracrRNA) to specifically bind to the target sequence. It is herein acknowledged and within the scope that single guide RNA (sgRNA) can be designed, which comprises the characteristics of both crRNA and tracrRNA.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “deaminase” as used herein refers to an enzyme that catalyzes the deamination reaction. In some embodiments of the present invention, the deaminase refers to a cytidine deaminase, which catalyzes the deamination of a cytidine or a deoxycytidine to a uracil or a deoxyuridine, respectively. In some other embodiments of the present invention, it refers to adenine deaminase. This enzyme catalyzes the hydrolytic deamination of adenosine to form inosine and deoxyadenosine to deoxyinosine.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “introduction” or “introduced” referring to a nucleic acid molecule (such as a plasmid, a linear nucleic acid fragment, RNA etc.) or protein into a plant means transforming the plant cell with the nucleic acid or protein so that the nucleic acid or protein can function in the plant cell.

As used herein, the term “transformation” includes stable transformation and transient transformation.

“Stable transformation” refers to introducing an exogenous nucleotide sequence into a plant genome, resulting in genetically stable inheritance. Once stably transformed, the exogenous nucleic acid sequence is stably integrated into the genome of the plant and any successive generations thereof.

“Transient transformation” refers to introducing a nucleic acid molecule or protein into a plant cell, performing its function without stable inheritance. In transient transformation, the exogenous nucleic acid sequence is not integrated into the plant genome.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1-12 refers to a variant of a sequence or part of a sequence which retains the biological function of the full non-variant allele (e.g. CsALS, CsCESA3, CsEPSPS and CsSF3B1 wild type allele) and hence has the activity of CsALS, CsCESA3, CsEPSPS and/or CsSF3B1 expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the herein identified gene sequences in Cannabis encoding herbicide resistance-related proteins, namely CsALS, CsCESA3, CsEPSPS and CsSF3B1 having the genomic nucleotide sequence as set forth in SEQ ID NOs: 1, 4, 7 and 10 respectively; coding sequence (CDS) as set forth in SEQ ID NOs: 2, 5, 8 and 11 respectively; and amino acid sequence as set forth in SEQ ID NOs: 3, 6, 9 and 12 respectively.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Cannabis plants of the present invention comprise homozygous configuration of at least one of the mutated genes encoding CsALS, CsCESA3, CsEPSPS and CsSF3B1, said mutated genes or variants confer herbicide resistance to the plant.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” “nucleic acid fragment” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene.

The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA. According to some further aspects of the present invention, these terms encompass a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, an “expression construct” or “expression cassette” refers to a vector suitable for expression of a nucleotide sequence of interest in a plant, such as a recombinant vector. “Expression” refers to the production of a functional product. For example, the expression of a nucleotide sequence may refer to transcription of the nucleotide sequence (such as transcribe to produce an mRNA or a functional RNA) and/or translation of RNA into a protein precursor or a mature protein. “Expression construct” of the invention may be a linear nucleic acid fragment, a circular plasmid, a viral vector, or, in some embodiments, an RNA that can be translated (such as an mRNA. According to further embodiments of the present invention, “expression construct” of the invention may comprise regulatory sequences and nucleotide sequences of interest that are derived from different sources, or regulatory sequences and nucleotide sequences of interest derived from the same source, but arranged in a manner different than that normally found in nature.

The term “regulatory sequence” or “regulatory element” are refer herein to nucleotide sequences located upstream (5′ non-coding sequences), within, or downstream (3′ non-coding sequences) of a coding sequence, and which influence or modulate or control the transcription, RNA processing or stability, or translation of the associated coding sequence. A plant expression regulatory element refers to a nucleotide sequence capable of controlling the transcription, RNA processing or stability or translation of a nucleotide sequence of interest in a plant. Regulatory sequences may include, but are not limited to, promoters, translation leader sequences, terminators, introns, and polyadenylation recognition sequences.

The term “promoter” refers to a nucleic acid fragment capable of controlling transcription of another nucleic acid fragment. In some embodiments of the invention, the promoter is a promoter capable of controlling gene transcription in a plant cell whether or not its origin is from a plant cell. The promoter may be a constitutive promoter or a tissue-specific promoter or a developmentally regulated promoter or an inducible promoter.

“Constitutive promoter” refers to a promoter that generally causes gene expression in most cell types in most circumstances. “Tissue-specific promoter” and “tissue-preferred promoter” are used interchangeably, and refer to a promoter that is expressed predominantly but not necessarily exclusively in one tissue or organ, but that may also be expressed in one specific cell or cell type. “Developmentally regulated promoter” refers to a promoter whose activity is determined by developmental events. “Inducible promoter” selectively expresses a DNA sequence operably linked to it in response to an endogenous or exogenous stimulus (such as environment, hormones, or chemical signals).

As used herein, the term “operably linked” means that a regulatory element (for example but not limited to, a promoter sequence, a transcription termination sequence etc.) is associated to a nucleic acid sequence (such as a coding sequence or an open reading frame), such that the transcription of the nucleotide sequence is controlled and regulated by the transcriptional regulatory element. Techniques for operably linking a regulatory element region to a nucleic acid molecule are known in the art.

The terms “peptide”, “polypeptide”, “protein” and “amino acid sequence” are used interchangeably herein and refer to amino acids in a polymeric form of any length, linked together by peptide bonds. In other words it encompass a polymer of amino acid residues. The terms apply also to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

According to other aspects of the invention, a “modified” or a “mutant” plant is a plant that has been altered compared to the naturally occurring wild type (WT) plant. Specifically, the endogenous nucleic acid sequences of HR-related gene homologs in Cannabis (nucleic acid sequences encoding CsALS, CsCESA3, CsEPSPS and CsSF3B1) have been altered compared to wild type sequences using mutagenesis and/or genome editing and/or base editing methods as described herein. This causes creation of precise mutations in the endogenous genes and thus production of proteins with amino acid change(s) that are necessary for the generation of enzymes insensitive to the corresponding herbicides, without loss of the enzymatic catalytic activity which might be essential for the cellular function.

It is within the scope of the current invention that such plants have an altered phenotype and show resistant or improved tolerance to the modified-enzyme corresponding herbicide, for example Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof.

According to further aspects of the present invention, the herbicide resistant trait is not conferred by the presence of transgenes expressed in Cannabis.

It should be noted that Cannabis plants of the invention are modified plants compared to wild type plants which comprise and express mutant alleles, genes or variants of at least one gene encoding CsALS, CsCESA3, CsEPSPS and/or CsSF3B1.

It is further noted that a wild type Cannabis plant is a plant that does not have any mutant CsALS, CsCESA3, CsEPSPS and/or CsSF3B1-encoding alleles.

It is further within the scope of the current invention that mutations in genes conferring herbicide resistance such as mutations in Cannabis genes encoding CsALS, CsCESA3, CsEPSPS and/or CsSF3B1, may be recessive, such that they are complemented by expression of a wild-type sequence. In alternative embodiments, they might have dominant or partially or co-dominant inheritance.

According to one embodiment, the present invention provides a modified Cannabis plant exhibiting herbicide resistance (HR) as compared to a Cannabis plant absent of such modification, wherein said modified plant comprises at least one genetically modified HR-related gene comprising at least one mutation conferring herbicide resistance to the plant.

According to a further embodiment, the present invention provides a modified Cannabis plant exhibiting herbicide resistance (HR) as compared to a Cannabis plant absent of such modification, wherein said plant comprises at least one base-editing driven modification in a HR-related gene in the plant genome, resulting in one or more nucleotide substitutions in the HR-related gene conferring herbicide resistance to the plant.

According to a further embodiment of the present invention, the at least one mutation is in a HR-related gene encoding for a Cannabis HR-related protein selected from acetolactate synthase (CsALS) 5-enolpyruvylshikimate-3-phosphate synthase (CsEPSPS), cellulose synthase A catalytic subunit 3 (CsCESA3), and splicing factor 3B subunit 1 (CsSF3B1).

According to a further embodiment of the present invention, the CsALS protein (wild-type) has an amino acid sequence as set forth in SEQ ID NO:3 or a functional variant thereof, and said protein is encoded by a genomic sequence as set forth in SEQ ID NO:1 or a functional variant thereof and/or by a coding sequence (CDS) as set forth in SEQ ID NO:2 or a functional variant thereof.

According to a further embodiment of the present invention, the CsCESA3 protein (wild-type) has an amino acid sequence as set forth in SEQ ID NO:6 or a functional variant thereof, and said protein is encoded by a genomic sequence as set forth in SEQ ID NO:4 or a functional variant thereof and/or by a coding sequence (CDS) as set forth in SEQ ID NO:5 or a functional variant thereof.

According to a further embodiment of the present invention, the CsEPSPS protein (wild-type) has an amino acid sequence as set forth in SEQ ID NO:9 or a functional variant thereof, and said protein is encoded by a genomic sequence as set forth in SEQ ID NO:7 or a functional variant thereof and/or by a coding sequence (CDS) as set forth in SEQ ID NO:8 or a functional variant thereof.

According to a further embodiment of the present invention, the CsSF3B1 protein (wild-type) has an amino acid sequence as set forth in SEQ ID NO:12 or a functional variant thereof, and said protein is encoded by a genomic sequence as set forth in SEQ ID NO:10 or a functional variant thereof and/or by a coding sequence (CDS) as set forth in SEQ ID NO:11 or a functional variant thereof.

According to a further embodiment of the present invention, the functional variant of any of the nucleic acid sequences or amino acid sequences mentioned above has at least 75% sequence identity to the corresponding nucleotide or amino acid sequence.

According to a further embodiment of the present invention, the mutated HR-related gene encodes a mutated CsALS protein comprising amino acid mutation at one or more positions selected from A118, P193, A201, W570 and S649 of SEQ ID NO: 3, said mutated protein confers herbicide resistance to a Cannabis plant.

According to a further embodiment of the present invention, the mutated HR-related gene encodes a mutated CsCESA3 protein comprising amino acid mutation at one or more positions selected from S998 and S1052 of SEQ ID NO: 6, said mutated protein confers herbicide resistance to a Cannabis plant.

According to a further embodiment of the present invention, the mutated HR-related gene encodes a mutated CsEPSPS protein comprising amino acid mutation at one or more positions selected from T181 and P185 of SEQ ID NO: 9, said mutated protein confers herbicide resistance to a Cannabis plant.

According to a further embodiment of the present invention, the mutated HR-related gene encodes a mutated CsSF3B1 protein comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant. According to a further embodiment of the present invention, the modified Cannabis plant comprises a base editing driven complex containing a nuclease-inactivated CRISPR/nuclease (Cas nickase or nCas) domain fused to a deaminase domain, and a gRNA comprising target sequence of the HR-related gene in the plant, said gRNA drive said complex to a target sequence in the herbicide resistance related gene in the plant so as to generate said one or more nucleotide substitutions.

According to a further embodiment of the present invention, the complex and said gRNA comprises at least one of the following: (a) a fusion protein comprising said nuclease-inactivated CRISPR/nuclease domain fused to a deaminase, and said guide RNA; (b) an expression construct comprising a nucleotide sequence encoding said nuclease-inactivated CRISPR/nuclease domain fused to a deaminase, and said guide RNA; (c) a fusion protein comprising said nuclease-inactivated CRISPR/nuclease domain fused to a deaminase, and an expression construct comprising a nucleotide sequence encoding said guide RNA; (d) an expression construct comprising a nucleotide sequence encoding said nuclease-inactivated CRISPR/nuclease domain fused to a deaminase domain, and an expression construct comprising a nucleotide sequence encoding said guide RNA; and (e) an expression construct comprising a nucleotide sequence encoding said nuclease-inactivated CRISPR/nuclease domain fused to a deaminase domain and a nucleotide sequence encoding said guide RNA.

It is further within the scope of the present invention that the base editing driven complex comprises at least one of: (a) a Cas9 nickase (nCas9) gene encoding an amino acid sequence as set forth in SEQ ID NO:14 fused to a deaminase and the target gene gRNA sequence, (b) a Ribonucleoprotein (RNP) complexes carrying nCas9 protein having an amino acid sequence as set forth in SEQ ID NO:14 fused to a deaminase protein and target gene gRNA sequence, or (c) RNP complex comprising the nCas9 protein having an amino acid sequence as set forth in SEQ ID NO:14 fused to a deaminase and the target gene gRNA sequence.

Thus, the present invention provides a base editing system or complex for generating herbicide-resistant plants. This system is designed for base editing of a herbicide resistance related gene in the genome of a Cannabis plant. The system may comprise at least one of the following (i) a base editing fusion protein, and a guide RNA; (ii) an expression construct comprising a nucleotide sequence encoding a base editing fusion protein, and a guide RNA; (iii) a base editing fusion protein, and an expression construct comprising a nucleotide sequence encoding a guide RNA; (iv) an expression construct comprising a nucleotide sequence encoding a base editing fusion protein, and an expression construct comprising a nucleotide sequence encoding a guide RNA; or (v) an expression construct comprising a nucleotide sequence encoding base editing fusion protein and a nucleotide sequence encoding guide RNA.

In some embodiments of the present invention, the base editing fusion protein or complex contains a nuclease-inactivated CRISPR/nuclease domain (such as nuclease-inactivated Cas9 domain or nCas9) and a deaminase domain.

In some embodiments, the guide RNA can target the base editing fusion protein to a target sequence in the herbicide resistance related gene in the Cannabis plant genome.

It is within the scope of the present invention that the herbicide resistance-related gene may be a gene encoding a protein having an important physiological activity in the Cannabis plant, which may be inhibited by the herbicide. Mutation in such herbicide-resistance-related gene may reverse the inhibition of the herbicide, but retain its physiological activity. Alternatively, the herbicide resistance related gene may encode a protein that is capable of degrading herbicides. Increasing the expression of such herbicide-associated gene or enhancing its degradation activity can result in increased resistance to herbicides.

According to some embodiments of the present invention, herbicide resistance-related genes include, but are not limited to, ALS (acetolactate synthase) gene (resistant to at least one of Chlorsulfuron, Bispyribac sodium, Imazamox, Tribenuron, Nicosulfuron, Mesosulfuron, Imazapic, Imazameth, Pyroxsulam, Flucarbazone-sodium, sulfonylurea, Imidazolidinone, etc.); EPSPS (5-enolpyruvate oxalate-3-phosphate synthase) gene (resistant to Glyphosate); CESA3 (cellulose synthase A catalytic subunit 3) gene (resistant to Cellulose biosynthesis-inhibiting chemical compound C17, etc.) and SF3B1 (splicing factor 3B subunit 1) gene (resistant to Herboxidiene (GEX1A) etc.).

In some embodiments, the guide RNA targets one or more of SEQ ID NOs: 1, 4, 7 and 10 encoding CsALS, CsCESA3, CsEPSPS and CsSF3B1, respectively.

It is noted that there is no specific limitation to the nuclease-inactivated CRISPR/nuclease that can be used in the present invention, provided that it retains the capability of targeting specific DNA under the guidance of gRNA, for example, those derived from Cas9, Cpf1 and the like can be used.

Examples of nuclease within the scope of the present invention include Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmrl, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi) and any combination thereof.

According to some aspects of the present invention, mutations in subdomains associate with DNA cleavage domain of Cas9 nuclease (the HNH nuclease subdomain and the RuvC subdomain) can inactivate Cas9 nuclease to form “nuclease-inactivated Cas9”. The nuclease-inactivated Cas9 retains DNA binding capacity directed by gRNA. Thus, when fused with an additional protein, the nuclease-inactivated Cas9 can simply target said additional protein to any DNA sequence through co-expression with appropriate guide RNA.

According to further aspects of the present invention, deaminase can catalyze the deamination of DNA nucleotide bases. In principle, if nuclease-inactivated Cas9 is fused with deaminase, the fusion protein can target a sequence in the genome of the Cannabis plant through the direction of a guide RNA. The DNA double strand is not cleaved due to the loss of Cas9 nuclease activity, whereas the deaminase domain in the fusion protein is capable of converting the cytidine or adenine of the single-strand DNA produced during the formation of the Cas9-guide RNA-DNA complex. Non-limiting examples of deaminase enzymes within the scope of the present invention may be apolipoprotein B mRNA editing complex (APOBEC) family deaminase, activation-induced cytidine deaminase (AID), APOBEC3G, CDA1, Activation-induced cytidine deaminase (AICDA), Cytidine deaminase (CDA), dCMP deaminase (DCTD), AMP deaminase (AMPD1), Adenosine Deaminase acting on tRNA (ADAT), Adenosine Deaminase acting on dsRNA (ADAR), Adenosine Deaminase acting on mononucleotides (ADA) or Guanine Deaminase (GDA).

According to some embodiments of the present invention, the nuclease-inactivated Cas9 of the present invention can be derived from Cas9 of different species, for example, derived from S. pyogenes Cas9 (SpCas9, the amino acid sequence of WT Cas9 is shown in SEQ ID NO:13). Mutations in both the HNH nuclease subdomain and the RuvC subdomain of the WT SpCas9 were generated (includes, for example, D10A and H840A mutations, see SEQ ID NO:14) which inactivate S. pyogenes Cas9 nuclease. Inactivation of one of the subdomains by mutation allows Cas9 to gain nickase activity, i.e., resulting in a Cas9 nickase (nCas9), for example, nCas9. having amino acid sequence as set forth in SEQ ID NO:14.

According to one embodiment, the nuclease-inactivated Cas9 is a Cas9 nickase that retains the cleavage activity of the HNH subdomain of Cas9, whereas the cleavage activity of the RuvC subdomain is inactivated.

In some embodiments of the invention, the deaminase domain is fused to the N-terminus of the nuclease-inactivated Cas9 domain. In some embodiments, the deaminase domain is fused to the C-terminus of the nuclease-inactivated Cas9 domain.

In some embodiments of the invention, the deaminase domain and the nuclease inactivated Cas9 domain are fused through a linker. The linker can be a non-functional amino acid sequence having no secondary or higher structure, which is 1 to 50 (for example, 1, 2, 3, 4, 5, 6, 7, 8, 9, 10, 11, 12, 13, 14, 15, 16, 17, 18, 19, 20, or 20-25, 25-50) or more amino acids in length.

In some embodiments of the invention, the base editing fusion protein of the invention further comprises a nuclear localization sequence (NLS). In general, one or more NLS in the base editing fusion protein or complex should have sufficient strength to drive the base editing fusion protein or complex in the nucleus of a plant cell sufficient for the base editing function. In general, the strength of the nuclear localization activity is determined by the number and position of NLS in the base editing fusion protein or complex.

In some embodiments of the present invention, the NLS of the base editing fusion protein or complex of the invention may be located at the N-terminus and/or the C-terminus. In some embodiments, the base editing fusion protein or complex comprises about 1, 2, 3, 4, 5, 6, 7, 8, 9, 10, or more NLSs. Where there are more than one NLS, each NLS may be selected as independently from other NLSs.

In general, the NLS consists of one or more short sequences of positively charged lysine or arginine exposed on the surface of a protein, but other types of NLS are also known in the art. Non-limiting examples of NLSs include KKRKV (nucleotide sequence 5′-AAGAAGAGAAAGGTC-3′), PKKKRKV (nucleotide sequence 5′-CCCAAGAAGAAGAGGAAGGTG-3′ or CCAAAGAAGAAGAGGAAGGTT), or SGGSPKKKRKV (nucleotide sequence 5′-TCGGGGGGGAGCCCAAAGAAGAAGCGGAAGGTG-3′).

In some aspects of the present invention, in order to obtain efficient expression in plants the nucleotide sequence encoding the base editing fusion protein or complex is codon optimized for the plant to be base edited. Codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments of the invention, the guide RNA is a single guide RNA (sgRNA). Methods of constructing suitable sgRNAs according to a given target sequence are known in the art.

In some embodiments of the invention, the nucleotide sequence encoding the base-editing fusion protein or complex and/or the nucleotide sequence encoding the guide RNA is operably linked to a plant expression regulatory element, such as a promoter.

Main aspects of the invention involve targeted mutagenesis methods, specifically genome editing and/or base editing, and exclude embodiments that are solely based on generating plants by traditional breeding methods. In a further embodiment of the current invention, as explained herein, the herbicide resistance at least one trait is not due to the presence of a transgene.

According to one embodiment of the present invention, HR-related Cannabis homologues such as CsALS, CsCESA3, CsEPSPS and CsSF3B1 have been identified in both genome and transcriptome in Cannabis.

The work inter alia described has important implications. The experiments are aimed at showing that CRISPR/Cas9 can be used to create heritable mutations in at least one of the genes encoding CsALS, CsCESA3, CsEPSPS and CsSF3B1 that result in desirable herbicide resistance phenotype in Cannabis.

It is further within the scope of the current invention that Cannabis genes, namely CsALS, CsCESA3, CsEPSPS and CsSF3B1-encoding genes having genomic nucleotide sequence as set forth in SEQ. ID. NO.: 1, 4, 7 and 10 respectively, were targeted using base editing complex and guide RNAs. The resultant mutated alleles have been identified.

The generated modified Cannabis plants could have improved agronomic value and are highly desirable due to their resistance to one or more herbicides used during Cannabis growth and cultivation.

It is further within the scope of the present invention that the harvest index (defined as the total yield per plant weight) of the HR modified plants is higher than that for wild type and/or Cannabis plants lacking the at least one modification in HR-related gene.

The plant of the invention includes plants wherein the plant is heterozygous for each of the mutations in at least one HR-related gene. In a preferred embodiment however, the plant is homozygous for the mutations. Progeny that is also homozygous can be generated from these plants according to methods known in the art.

It is further within the scope that variants of a particular CsALS, CsCESA3, CsEPSPS or CsSF3B1 nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant CsALS, CsCESA3, CsEPSPS and CsSF3B1 nucleotide sequence allele as shown in SEQ ID NO 1, 4, 7 and 10; and/or SEQ ID NO 2, 5, 8 and 11; and/or SEQ ID NO 3, 6, 9 and 12 respectively. Sequence alignment programs to determine sequence identity are well known in the art.

Also, the various aspects of the invention encompass not only a CsALS, CsCESA3, CsEPSPS or CsSF3B1 nucleic acid sequence or amino acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, e.g., enzymatic activity and/or herbicide resistance trait.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein—RNP's) in order to bypass the need for in vivo transcription and translation of the Cas9+gRNA plasmid in planta to achieve gene editing.

It is also possible to create a genome edited/base edited plant and use it as a rootstock. Then, the Cas protein and gRNA can be transported via the vasculature system to the top of the plant and create the genome editing event in the scion.

It is further within the scope that herbicide resistance traits in Cannabis plants are herein produced by generating gRNA with homology to a specific site of predetermined genes in the Cannabis genome i.e. genes encoding CsALS, CsCESA3, CsEPSPS and/or CsSF3B1, sub cloning this gRNA into a plasmid containing the Cas9 gene, and insertion of the plasmid into the Cannabis plant cells. In this way site specific mutations in at least one of the genes encoding CsALS, CsCESA3, CsEPSPS and/or CsSF3B1 are generated thus effectively creating herbicide resistance Cannabis plants.

Thus, in another aspect, the present invention provides for the first time method for producing herbicide-resistant (HR) Cannabis plants by genome editing technique (e.g. base editing).

According to one embodiment, the method for producing a herbicide-resistant (HR) Cannabis plant comprises steps of genetically modifying at least one HR-related gene by introducing at least one mutation within said at least one HR-related gene so as to confer herbicide resistance to the plant.

According to a further embodiment, the method for producing a herbicide-resistant (HR) Cannabis plant comprises steps of introducing by base-editing, a modification in a HR-related gene in the plant genome, so as to generate one or more nucleotide substitutions in the HR-related gene, wherein said one or more nucleotide substitutions confers herbicide resistance to the Cannabis plant.

In some embodiments, the method further comprises steps of screening the plants for herbicide resistance.

In some embodiments of the method described above, the steps of introducing by base-editing a modification in a HR-related gene in the plant genome, comprising introducing into the plant a complex containing a nuclease-inactivated CRISPR/nuclease domain fused to a deaminase domain, and a gRNA comprising target sequence of the HR-related gene in the plant. In this way, the gRNA drives said complex to the target sequence in the herbicide resistance related gene in the plant so as to generate the one or more nucleotide substitutions conferring herbicide resistance to the plant.

According to further embodiments of the method of the present invention, the complex and said gRNA comprises at least one of the following: (a) a fusion protein comprising said nuclease-inactivated CRISPR/nuclease domain fused to a deaminase, and said guide RNA; (b) an expression construct comprising a nucleotide sequence encoding said nuclease-inactivated CRISPR/nuclease domain fused to a deaminase, and said guide RNA; (c) a fusion protein comprising said nuclease-inactivated CRISPR/nuclease domain fused to a deaminase, and an expression construct comprising a nucleotide sequence encoding said guide RNA; (d) an expression construct comprising a nucleotide sequence encoding said nuclease-inactivated CRISPR/nuclease domain fused to a deaminase domain, and an expression construct comprising a nucleotide sequence encoding said guide RNA; and (e) an expression construct comprising a nucleotide sequence encoding said nuclease-inactivated CRISPR/nuclease domain fused to a deaminase domain and a nucleotide sequence encoding said guide RNA.

According to some further embodiments of the method of the present invention, at least one mutation is introduced into a HR-related gene encoding for a Cannabis HR-related protein selected from acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1).

According to some further embodiments of the method of the present invention, the CsALS protein has an amino acid sequence as set forth in SEQ ID NO:3 or a functional variant thereof, and said protein is encoded by a genomic sequence as set forth in SEQ ID NO:1 or a functional variant thereof and/or by a coding sequence (CDS) as set forth in SEQ ID NO:2 or a functional variant thereof.

According to some further embodiments of the method of the present invention, the CsCESA3 protein has an amino acid sequence as set forth in SEQ ID NO:6 or a functional variant thereof, and said protein is encoded by a genomic sequence as set forth in SEQ ID NO:4 or a functional variant thereof and/or by a coding sequence (CDS) as set forth in SEQ ID NO:5 or a functional variant thereof.

According to some further embodiments of the method of the present invention, the CsEPSPS protein has an amino acid sequence as set forth in SEQ ID NO:9 or a functional variant thereof, and said protein is encoded by a genomic sequence as set forth in SEQ ID NO:7 or a functional variant thereof and/or by a coding sequence (CDS) as set forth in SEQ ID NO:8 or a functional variant thereof.

According to some further embodiments of the method of the present invention the CsSF3B1 protein has an amino acid sequence as set forth in SEQ ID NO:12 or a functional variant thereof, and said protein is encoded by a genomic sequence as set forth in SEQ ID NO:10 or a functional variant thereof and/or by a coding sequence (CDS) as set forth in SEQ ID NO:11 or a functional variant thereof.

According to some further embodiments of the method of the present invention the functional variant has at least 75% sequence identity to the corresponding nucleotide or amino acid sequence.

According to some further embodiments of the method of the present invention, the mutated HR-related gene encodes a mutated CsALS protein, comprising amino acid mutation at one or more positions selected from A118X, P193X, A201X, W570X and S649X of SEQ ID NO: 3, said mutated protein confers herbicide resistance to a Cannabis plant (X represents any amino acid substitution).

According to some further embodiments of the method of the present invention, the mutated HR-related gene encodes a mutated CsCESA3 protein, comprising amino acid mutation at one or more positions selected from S998X and S1052X of SEQ ID NO: 6, said mutated protein confers herbicide resistance to a Cannabis plant (X represents any amino acid substitution).

According to some further embodiments of the method of the present invention, the mutated HR-related gene encodes a mutated CsEPSPS protein, comprising amino acid mutation at one or more positions selected from T181X and P185X of SEQ ID NO: 9, said mutated protein confers herbicide resistance to a Cannabis plant (X represents any amino acid substitution).

According to some further embodiments of the method of the present invention, the mutated HR-related gene encodes a mutated CsSF3B1 protein, comprising amino acid mutation at one or more positions selected from SGR4 (K1033X and/or K1034X and/or G1035X), SGR5 (H1032X), and SGR6 (H1032X and/or A1049X) of SEQ ID NO: 12, said variant confers herbicide resistance to a Cannabis plant (X represents any amino acid substitution).

In another aspect, the present invention provides a method for producing a herbicide-resistant Cannabis plant, comprising introducing into the plant a system of the present invention for base-editing a herbicide resistance-related gene in the Cannabis plant genome, thereby the guide RNA targets the base-editing fusion protein or complex to a target sequence of a herbicide resistance-related gene in the Cannabis plant, resulting in one or more nucleotide substitutions in the target sequence.

In some embodiments, the herbicide resistance-related gene encodes a herbicide resistance-related protein selected from the group consisting of CsALS, CsCESA3, CsEPSPS and/or CsSF3B1.

In some embodiments, the nucleotide substitution is a C to T substitution. In some embodiments, the nucleotide substitution is a C to A or C to G substitution. In some embodiments, the nucleotide substitution in located in the non-coding region in the herbicide resistance related gene, such as expression regulation regions (e.g. promoter region). In some embodiments, the nucleotide substitution results in amino acid substitution in the herbicide resistance protein encoded by the gene. In some embodiments, the nucleotide substitution and/or amino acid substitution confer herbicide resistance to the Cannabis plant.

In some embodiments of the present invention, the nucleotide substitution and/or amino acid substitution that confer herbicide resistance to a plant may be any known substitution that confers herbicide resistance to a plant in a herbicide resistance-related gene. By the method of the present invention, single mutations, double mutations or multiple mutations capable of conferring herbicide resistance can be created in situ in plants without the need of transgene. The mutations may be known in the art or may be newly identified (specific to Cannabis) by the methods of the present invention.

The present invention provides a method for producing a herbicide-resistant plant, comprising modifying at least one of CsALS, CsCESA3, CsEPSPS and/or CsSF3B1 genes in a Cannabis plant (SEQ ID NOs: 1, 4, 7 or 10 respectively) by the base-editing method of the present invention, resulting in one or more amino acid mutations in one or more of the aforementioned herbicide resistance-related genes which confer herbicide resistance to the plant.

In some embodiments, the CsALS wild-type genomic, CDS and amino acid sequence is shown in SEQ ID No: 1, 2 and 3, respectively.

In some embodiments, the CsCESA3 wild-type genomic, CDS and amino acid sequence is shown in SEQ ID No: 4, 5 and 6, respectively.

In some embodiments, the CsEPSPS wild-type genomic, CDS and amino acid sequence is shown in SEQ ID No: 7, 8 and 9, respectively.

In some embodiments, the CsSF3B1 wild-type genomic, CDS and amino acid sequence is shown in SEQ ID No: 10, 11 and 12, respectively.

The present invention provides a method for producing a herbicide-resistant plant, comprising modifying a CsALS gene in a Cannabis plant by the base editing method of the present invention, resulting in one or more amino acid mutations in CsALS which confer herbicide resistance to the plant.

It is within the scope of the present invention that the mutated or modified CsALS protein, comprises amino acid mutation at one or more positions selected from A118X, P193X, A201X, W570X and S649X of SEQ ID NO: 3, said mutated protein confers herbicide resistance to a Cannabis plant (X represents any amino acid substitution). The present invention provides a method for producing a herbicide-resistant plant, comprising modifying a CsCESA3 gene in a Cannabis plant by the base editing method of the present invention, resulting in one or more amino acid mutations in CsCESA3 which confer herbicide resistance to the plant.

It is within the scope of the present invention that the mutated or modified CsCESA3 protein comprises amino acid mutation at one or more positions selected from S998X and S1052X of SEQ ID NO: 6, said mutated protein confers herbicide resistance to a Cannabis plant (X represents any amino acid substitution).

The present invention provides a method for producing a herbicide-resistant plant, comprising modifying a CsEPSPS gene in a Cannabis plant by the base editing method of the present invention, resulting in one or more amino acid mutations in CsEPSPS which confer herbicide resistance to the plant.

It is within the scope of the present invention that the mutated or modified CsEPSPS protein comprises amino acid mutation at one or more positions selected from T181X and P185X of SEQ ID NO: 9, said mutated protein confers herbicide resistance to a Cannabis plant (X represents any amino acid substitution).

The present invention provides a method for producing a herbicide-resistant plant, comprising modifying a CsSF3B1 gene in a Cannabis plant by the base editing method of the present invention, resulting in one or more amino acid mutations in CsSF3B1 which confer herbicide resistance to the plant.

It is within the scope of the present invention that the mutated or modified CsSF3B1 protein comprises amino acid mutation at one or more positions selected from SGR4 (K1033X and/or K1034X and/or G1035X), SGR5 (H1032X), and SGR6 (H1032X and/or A1049X) of SEQ ID NO: 12, said variant confers herbicide resistance to a Cannabis plant (X represents any amino acid substitution).

It is noted that the design of the target sequence that can be recognized and targeted by a Cas9 and guide RNA complex is within the technical skills of one of ordinary skill in the art. In general, the target sequence is a sequence that is complementary to a leader sequence of about 20 nucleotides comprised in guide RNA, and the 3-end of which is immediately adjacent to the protospacer adjacent motif (PAM) NGG.

In some embodiments of the methods of the invention, further comprises screening plants having the desired nucleotide substitutions. Nucleotide substitutions in plants can be detected by T7EI, PCR/RE or sequencing methods known in the art.

In the methods of the invention, the base editing system and method can be introduced into Cannabis plants by various methods well known to people skilled in the art or variations thereof adapted to the Cannabis plant. Methods that can be used to introduce the base editing system of the present invention into Cannabis plants include but not limited to particle bombardment, PEG-mediated protoplast transformation, Agrobacterium-mediated transformation, plant virus-mediated transformation, pollen tube approach, and ovary injection approach. In some embodiments, the base editing system is introduced into Cannabis plants by transient transformation.

In the methods of the present invention, modification of the target sequence can be accomplished by introducing or producing the base editing fusion protein or complex and guide RNA in plant cells, and the modification can be stably inherited without the need of stably transformation of plants with the base editing system. This avoids potential off-target effects of a stable base editing system, and also avoids the integration of exogenous nucleotide sequences into the plant genome, and thereby resulting in safety regulatory issues.

In some preferred embodiments, the introduction is performed in the absence of a selective pressure, thereby avoiding the integration of exogenous nucleotide sequences in the plant genome.

In some embodiments, the introduction comprises transforming the base editing system of the invention into isolated Cannabis cells or tissues, and then regenerating the transformed plant cells or tissues into an intact Cannabis plant.

In other embodiments, the base editing system of the present invention can be transformed to a particular site on an intact plant, such as leaf, shoot tip, pollen tube or hypocotyl.

In some embodiments, the herbicide-resistant plant is transgene-free

In some embodiments of the invention, the method further comprises obtaining progeny of the herbicide-resistant Cannabis plant.

In another aspect, the present invention also provides a herbicide-resistant Cannabis plant or progeny or parts thereof, wherein the plant is obtained by the above-described method of the present invention. In some embodiments, the herbicide-resistant Cannabis plant is transgene-free.

In another aspect, the present invention also provides a method comprising crossing a first herbicide-resistant Cannabis plant obtained by the above-described method of the present invention with a second Cannabis plant having no herbicide resistance, and thereby introducing the herbicide resistance into the second plant. This also may be achieved by transforming the Cannabis HR-related variant produced by the method of the present invention into a non-HR Cannabis plant.

The present invention also encompasses the herbicide-resistant Cannabis plant or progeny thereof obtained by the method of the present invention.

According to further aspects, the present invention provides a method of identifying a variant of a Cannabis herbicide-resistance (HR) related protein, wherein said variant is capable of conferring herbicide resistance to a Cannabis plant. The aforementioned method comprising: (a) generating a HR Cannabis plant by the method as defined in any of the above; and (b) determining the sequence of the HR-related gene and/or the encoded HR-related protein in the resulting herbicide resistant plant, thereby identifying the sequence of the variant.

In order to understand the invention and to see how it may be implemented in practice, a plurality of preferred embodiments will now be described, by way of non-limiting example only, with reference to the following examples.

### Example 1

Production of herbicide resistant Cannabis plants by targeted base-editing Production of Cannabis lines with mutated herbicide resistance related genes may be achieved by at least one of the following breeding/cultivation schemes:

### Scheme 1

line stabilization by self pollination
Generation of F6 parental lines
Base editing of parental lines
Crossing edited parental lines to generate an F1 hybrid plant

### Scheme 2

Identifying genes/alleles of interest
Designing gRNA
Transformation of plants with Cas9+gRNA constructs
Screening and identifying editing events
Genome editing of parental lines

According to specific embodiments, Cannabis genes identified as involved in herbicide resistance (HR) are targeted to develop HR Cannabis. These genes encode proteins including CsALS, CsCESA3, CsEPSPS and CsSF3B1. In the next stage amino acid change(s) that are necessary for the generation of enzymes insensitive to the corresponding herbicides were revealed. The targeted amino acid changes should show improved herbicide tolerance without loss of the enzymatic catalytic activity. To accomplish programmable base-editing, deaminase enzyme (e.g. adenine deaminase or cytidine deaminase) was fused to a catalytically impaired CRISPR/Cas mutant (i.e. Cas9 nickase or nCas9), leading to A-to-G or C-to-T substitution without the introduction of a double-strand break (DSB) in the DNA. These base editors drive the nCas9-gRNA complex to the target locus and enable deamination on the non-complementary strand.

It is noted that line stabilization may be performed by the following:
Induction of male flowering on female (XX) plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Cannabis strains.

According to a further aspect of the current invention, shortening line stabilization is performed by Doubled Haploids (DH). More specifically, the CRISPR-Cas9 (or CRISPR-nCas9) system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program
Genotyping markers—germplasm used in the current invention is genotyped using molecular markers, in order to allow a more efficient breeding process and identification of the HR-related genes one or more editing events.

It is further within the scope of the current invention that allele and genetic variation is analyzed for the Cannabis strains used.

Reference is now made to optional stages that have been used for the production of herbicide resistance Cannabis plants by genome/base editing:

Stage 1: Identifying Cannabis sativa (C. sativa) and/or C. indica and/or C. ruderalis HR-Related Gene Orthologues.

In this example, Cannabis orthologues for herbicide resistance-related genes such as ALS, CESA3, EPSPS and SF3B1 (namely CsALS, CsCESA3, CsEPSPS and CsSF3B1 respectively) were identified. These homologous genes have been sequenced and mapped.

The Cannabis gene encoding CsALS has been mapped to NC_044372.1:7605596-7608040 Cannabis sativa chromosome 3 and has a genomic sequence as set forth in SEQ ID NO:1. The CsALS encoding gene has a coding sequence (CDS) as set forth in SEQ ID NO:2 and it encodes an amino acid sequence as set forth in SEQ ID NO:3.

The Cannabis gene encoding CsCESA3 has been mapped to CsCESA3 NC_044372.1:1649106-1654442 Cannabis sativa chromosome 3 and has a genomic sequence as set forth in SEQ ID NO:4. The CsCESA3 encoding gene has a coding sequence (CDS) as set forth in SEQ ID NO:5 and it encodes an amino acid sequence as set forth in SEQ ID NO:6.

The Cannabis gene encoding CsEPSPS has been mapped to NC_044371.1:71089167-71093882 Cannabis sativa chromosome 1 and has a genomic sequence as set forth in SEQ ID NO:7. The CsEPSPS encoding gene has a coding sequence (CDS) as set forth in SEQ ID NO:8 and it encodes an amino acid sequence as set forth in SEQ ID NO:9.

The Cannabis gene encoding CsSF3B1 has been mapped to CsSF3B1 NC_044373.1:83909151-83913459 Cannabis sativa chromosome 4 and has a genomic sequence as set forth in SEQ ID NO:10. The CsSF3B1 encoding gene has a coding sequence (CDS) as set forth in SEQ ID NO:11 and it encodes an amino acid sequence as set forth in SEQ ID NO:12.

Reference is made to Table 1 presenting a summary of the sequences and corresponding SEQ ID Nos within the scope of the current invention.

TABLE 1
Summary of sequences within the scope of the present invention

| Sequence type | CsALS | CsCESA3 | CsEPSPS | CsSF3B1 | Cas9 | nCas9 |
|---|---|---|---|---|---|---|
| Genomic | SEQ ID | SEQ ID | SEQ ID | SEQ ID | | |
| sequences | NO: 1 | NO: 4 | NO: 7 | NO: 10 | | |
| Coding | SEQ ID | SEQ ID | SEQ ID | SEQ ID | | |
| sequences | NO: 2 | NO: 5 | NO: 8 | NO: 11 | | |
| (CDS) | | | | | | |
| Amino acid | SEQ ID | SEQ ID | SEQ ID | SEQ ID | SEQ ID | SEQ ID |
| sequences | NO: 3 | NO: 6 | NO: 9 | NO: 12 | NO: 13 | NO: 14 |

### Stage 2: Construction of Base Editing Vectors or Constructs

Base editing vectors targeting herbicide resistance-related genes such as CsALS, CsCESA3, CsEPSPS and CsSF3B1 for Cannabis plant were constructed.

In addition, gRNA molecules corresponding to the sequence targeted for editing, i.e. sequences of each of the genes encoding CsALS, CsCESA3, CsEPSPS and CsSF3B1 were designed and synthesized.

It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different CsALS, CsCESA3, CsEPSPS and CsSF3B1 homologues of different Cannabis strains.

According to a further embodiment, ‘PAM’ (Protospacer Adjacent Motif) sequences, which is a 2-6 base pair DNA sequence immediately following the DNA sequence targeted by the Cas (e.g. Cas9) nuclease in the CRISPR bacterial adaptive immune system.

The gRNA molecules have been cloned into suitable vectors and their sequence has been verified. In addition different Cas9 versions have been analyzed for optimal compatibility between the Cas9 protein activity and the gRNA molecule in the Cannabis plant.

The efficiency of the designed gRNA molecules have been validated by transiently transforming Cannabis tissue culture. A plasmid carrying a gRNA sequence together with the Cas9 gene has been transformed into Cannabis protoplasts. The protoplast cells have been grown for a short period of time and then were analyzed for existence of genome editing events. The positive constructs have been subjected to the herein established stable transformation protocol into Cannabis plant tissue for producing genome edited Cannabis plants in at least one gene encoding CsALS, CsCESA3, CsEPSPS and/or CsSF3B1.

The construct for base editing in Cannabis comprises one of the following options:
1. a fusion protein comprising nuclease-inactivated CRISPR/nuclease domain fused to a deaminase, and said guide RNA;
2. an expression construct comprising a nucleotide sequence encoding said nuclease-inactivated CRISPR/nuclease domain fused to a deaminase, and said guide RNA;
3. a fusion protein comprising said nuclease-inactivated CRISPR/nuclease domain fused to a deaminase, and an expression construct comprising a nucleotide sequence encoding said guide RNA;
4. an expression construct comprising a nucleotide sequence encoding said nuclease-inactivated CRISPR/nuclease domain fused to a deaminase domain, and an expression construct comprising a nucleotide sequence encoding said guide RNA;
5. an expression construct comprising a nucleotide sequence encoding said nuclease-inactivated CRISPR/nuclease domain fused to a deaminase domain and a nucleotide sequence encoding said guide RNA.
   Stage 3: Transforming Cannabis Plants i.e. Using Agrobacterium-Mediated or Biolistics (Gene Gun) Methods.

For Agrobacterium and biolistics, a DNA plasmid carrying (Cas9+deaminase+gene specific gRNA) can be used. A vector containing a selection marker, Cas9 gene fused to a deaminase and relevant target gene specific gRNA sequence is constructed. For biolistics, Ribonucleoprotein (RNP) complexes carrying (Cas9 protein fused to a deaminase protein and target gene specific gRNA) are used. RNP complexes are created by mixing the Cas9 protein fused to a deaminase with relevant gene specific gRNA's.

According to some embodiments of the present invention, transformation of various Cannabis tissues was performed using particle bombardment of:
DNA vectors
Ribonucleoprotein complex (RNP's)

According to further embodiments of the present invention, transformation of various Cannabis tissues was performed using Agrobacterium-mediated transformation (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed (see for example WO2020170251 and WO2020035869 incorporated herein by reference). Various Cannabis tissues have been successfully transiently transformed using biolistics system. Transformation has been performed into calli, leaves, axillary buds and cotyledons of Cannabis.

According to further embodiments of the present invention, additional transformation tools were used in Cannabis, including, but not limited to:
Protoplast PEG transformation
Extend RNP use
Directed editing screening using fluorescent tags
Electroporation

### Stage 4: Regeneration in Tissue-Culture.

When transforming DNA constructs into the plant, antibiotics is used for selection of positive transformed plants (see for example WO2020170251 and WO2020035869 incorporated herein by reference).

Stage 5: Screening and Selection for Transformed Cannabis Plants. Establishing Resistance Screening Conditions for Transformed Plants

Once regenerated plants appear (e.g. in tissue culture), DNA is extracted from leaf sample of the transformed plant and PCR is performed using primers flanking the regions targeted for base editing. PCR products are then digested with enzymes recognizing the restriction site near the original gRNA sequence. If editing event occurred, the restriction site will be disrupted and the PCR product will not be cleaved. No editing event will result in a cleaved PCR product.

Screening for nuclease-inactivated CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

The analysis of the transformed plants encompass the following steps:
1) Amplicon is isolated from two exemplified Cannabis strains by primers flanking the sequence of the gene of interest targeted by the predesigned sgRNA.
2) RNP complex is incubated with the isolated amplicon.
3) The reaction mix is then loaded on agarose gel to evaluate Cas9 cleavage activity at the target site.

### Stage 6: Screening and Identification of Herbicide Resistant Cannabis Plants

The transformed plants obtained in step 5 were grown on the corresponding herbicide screening medium and the phenotypes were observed and the resistant plants were selected.

Herbicides used for screening include, but are not limited to, Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof. The herbicide concentrations used for screening were the minimum conventions found as inhibiting plant growth.

DNA was extracted from the resistant plants, and the base-editing mutations of the target genes were assessed by PCR and sequencing techniques. As a result mutations in Cannabis-endogenous proteins CsALS, CsCESA3, CsEPSPS and/or CsSF3B1 are identified as conferring herbicide-resistance.

1. A method of producing a herbicide-resistant (HR) Cannabis plant, wherein said method comprises steps of genetically modifying by base-editing at least one HR-related gene by introducing at least one mutation within said at least one HR-related gene so as to generate one or more nucleotide substitutions in the HR-related gene, said one or more nucleotide substitutions confer herbicide resistance to the Cannabis plant.

2. The method according to claim 1, wherein said one or more nucleotide substitutions is introduced using a base editing driven complex comprising at least one of: (a) a Cas9 nickase (nCas9) gene encoding an amino acid sequence as set forth in SEQ ID NO:14 fused to a deaminase and the target gene gRNA sequence, (b) a Ribonucleoprotein (RNP) complexes carrying nCas9 protein having an amino acid sequence as set forth in SEQ ID NO:14 fused to a deaminase protein and target gene gRNA sequence, or (c) RNP complex comprising the nCas9 protein having an amino acid sequence as set forth in SEQ ID NO:14 fused to a deaminase and the target gene gRNA sequence.

3. The method according to claim 1, wherein the at least one mutation is introduced into a HR-related gene encoding for a Cannabis HR-related protein selected from acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1).

4. The method according to claim 3, wherein at least one of the following holds true:
a. said CsALS protein has an amino acid sequence comprising at least 75% sequence identity to the sequence as set forth in SEQ ID NO:3, and said protein is encoded by a genomic sequence comprising at least 75% sequence identity to the sequence as set forth in SEQ ID NO:1 and/or by a coding sequence (CDS) comprising at least 75% sequence identity to the sequence as set forth in SEQ ID NO:2;
b. said CsCESA3 protein has an amino acid sequence comprising at least 75% sequence identity to the sequence as set forth in SEQ ID NO:6, and said protein is encoded by a genomic sequence comprising at least 75% sequence identity to the sequence as set forth in SEQ ID NO:4 and/or by a coding sequence (CDS) comprising at least 75% sequence identity to the sequence as set forth in SEQ ID NO:5;
c. said CsEPSPS protein has an amino acid sequence comprising at least 75% sequence identity to the sequence as set forth in SEQ ID NO:9, and said protein is encoded by a genomic sequence comprising at least 75% sequence identity to the sequence as set forth in SEQ ID NO:7 and/or by a coding sequence (CDS) comprising at least 75% sequence identity to the sequence as set forth in SEQ ID NO:8; and
d. said CsSF3B1 protein has an amino acid sequence comprising at least 75% sequence identity to the sequence as set forth in SEQ ID NO:12, and said protein is encoded by a genomic sequence comprising at least 75% sequence identity to the sequence as set forth in SEQ ID NO:10 and/or by a coding sequence (CDS) comprising at least 75% sequence identity to the sequence as set forth in SEQ ID NO:11.

5. (canceled)

6. (canceled)

7. (canceled)

8. The method according to claim 1, wherein the mutated HR-related gene encodes at least one of the following:
e. a mutated CsALS protein, comprising amino acid mutation at one or more positions selected from A118, P193, A201, W570 and S649 of SEQ ID NO: 3, said mutated protein confers herbicide resistance to a Cannabis plant;
f. a mutated CsCESA3 protein, comprising amino acid mutation at one or more positions selected from S998 and S1052 of SEQ ID NO: 6, said mutated protein confers herbicide resistance to a Cannabis plant;
g. a mutated CsEPSPS protein, comprising amino acid mutation at one or more positions selected from T181 and P185 of SEQ ID NO: 9, said mutated protein confers herbicide resistance to a Cannabis plant; and
h. a mutated CsSF3B1 protein, comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant.

9. (canceled)

10. (canceled)

11. (canceled)

12. The method according to claim 2 wherein said gRNA targets one or more of SEQ ID NOs: 1, 4, 7 and 10 or a portion thereof.

13. The method according to claim 1, wherein said at least one mutation is in the coding region of said gene, in the regulatory region of said gene, or an epigenetic factor.

14. The method according to claim 1, wherein said plant comprises at least one variant of a HR-related gene encoding a variant of a protein selected from the group consisting of CsALS, CsEPSPS, CsCESA3, and CsSF3B1 and any combination thereof, said protein variant is capable of conferring herbicide resistance to a Cannabis plant.

15. The method according to claim 14, wherein said protein variant is selected from the group consisting of:
i. CsALS variant comprising amino acid mutation at one of more positions selected from A118X, P193X, A201X, W570X and or S649X of SEQ ID NO: 3, said variant confers herbicide resistance to a Cannabis plant;
j. CsCESA3 variant comprising amino acid mutation at one of more positions selected from S998X and S1052X of SEQ ID NO: 6, said variant confers herbicide resistance to a Cannabis plant;
k. CsEPSPS variant comprising amino acid mutation at one of more positions selected from T181X and P185X of SEQ ID NO: 9, said variant confers herbicide resistance to a Cannabis plant; and
l. CsSF3B1 variant comprising amino acid mutation at one of more positions selected from SGR4 (K1033X/K1034X/G1035X), SGR5 (H1032X), and SGR6 (H1032X/A1049X) of SEQ ID NO: 12, said variant confers herbicide resistance to a Cannabis plant.

16. The method according to claim 1, wherein said plant is resistant to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof.

17. The method according to claim 1 wherein said plant is characterized by enhanced tolerance to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof, as compared to a Cannabis plant lacking said at least one modification.

18. The method according to claim 1, comprising steps of screening the modified plant for herbicide resistance.

19. A modified Cannabis plant exhibiting herbicide resistance (HR) as compared to a Cannabis plant absent of such modification, wherein said modified plant is produced by the method according to claim 1.

20. The modified Cannabis plant according to claim 19, wherein at least one of the following holds true:
m. the at least one genetically modified HR-related gene comprises one or more base-editing driven nucleotide substitutions, resulting in a mutated HR-related gene conferring herbicide resistance to the plant;
n. the at least one mutation is in a HR-related gene encoding for a Cannabis HR-related protein selected from acetolactate synthase (CsALS), 5-enolpyruvylshikimate-3-phosphate synthase (CsEPSPS), cellulose synthase A catalytic subunit 3 (CsCESA3), and splicing factor 3B subunit 1 (CsSF3B1);
o. the mutated HR-related gene encodes a mutated CsALS protein comprising amino acid mutation at one or more positions selected from A118, P193, A201, W570 and S649 of SEQ ID NO: 3, said mutated protein confers herbicide resistance to a Cannabis plant;
p. the mutated HR-related gene encodes a mutated CsCESA3 protein comprising amino acid mutation at one or more positions selected from S998 and S1052 of SEQ ID NO: 6, said mutated protein confers herbicide resistance to a Cannabis plant;
q. the mutated HR-related gene encodes a mutated CsEPSPS protein comprising amino acid mutation at one or more positions selected from T181 and P185 of SEQ ID NO: 9, said mutated protein confers herbicide resistance to a Cannabis plant;
r. the mutated HR-related gene encodes a mutated CsSF3B1 protein comprising amino acid mutation at one or more positions selected from SGR4 (K1033 and/or K1034 and/or G1035), SGR5 (H1032), and SGR6 (H1032 and/or A1049) of SEQ ID NO: 12, said variant confers herbicide resistance to a Cannabis plant;
s. said plant comprises a base editing driven complex containing a nuclease-inactivated CRISPR/nuclease domain (Cas nickase or nCas) fused to a deaminase domain, and a gRNA comprising target sequence of the HR-related gene in the plant, said complex is driven by said gRNA to a target sequence in the herbicide resistance related gene in the plant so as to generate said one or more nucleotide substitutions;
t. said plant is homozygous for said at least one mutated allele;
u. said at least one mutation is in the coding region of said gene, in the regulatory region of said gene, or an epigenetic factor;
v. said plant comprises at least one variant of a HR-related gene encoding a variant of a protein selected from the group consisting of CsALS, CsEPSPS, CsCESA3, and CsSF3B1 and any combination thereof, said protein variant is capable of conferring herbicide resistance to a Cannabis plant;
w. said plant is resistant to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1A) and any combination thereof; and
x. said plant is characterized by enhanced tolerance to at least one herbicide selected from the group consisting of Chlorsulfuron, Bispyribac sodium, Imazamox, Tribenuron, Nicosulfuron, Mesosulfuron, Imazapic, Glyphosate, Cellulose biosynthesis-inhibiting chemical compound such as C17, Herboxidiene (GEX1 A) and any combination thereof, as compared to a Cannabis plant lacking said at least one modification.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A Cannabis plant, plant part or plant cell according to claim 19 wherein said plant is a transgene-free herbicide-resistant plant.

41. A plant part, plant cell, tissue culture of regenerable cells, protoplasts, callus or plant seed obtained from the modified Cannabis plant according to claim 19.

42. A method of identifying a HR Cannabis plant comprising variant or mutant of a Cannabis herbicide-resistance (HR) related protein, wherein said variant or mutant is capable of conferring herbicide resistance to a Cannabis plant, said method comprising:
y. optionally, generating a HR Cannabis plant by the method of claim 1; and
z. determining the sequence of the HR-related gene and/or the encoded HR-related protein in the herbicide resistant plant, thereby identifying the sequence of the variant or mutant,
wherein said HR-related protein variant or mutant is selected from the group consisting of:
i. variant of SEQ ID NO:3 comprising amino acid mutation at one of more positions selected from A118X, P193X, A201X, W570X and or S649X, said variant confers herbicide resistance to a Cannabis plant;
ii. variant of SEQ ID NO:6 comprising amino acid mutation at one of more positions selected from S998X and S1052X, said variant confers herbicide resistance to a Cannabis plant;
iii. variant of SEQ ID NO:9 comprising amino acid mutation at one of more positions selected from T181X and P185X, said variant confers herbicide resistance to a Cannabis plant; and
iv. variant of SEQ ID NO:12 comprising amino acid mutation at one of more positions selected from SGR4 (K1033X/K1034X/G1035X), SGR5 (H1032X), and SGR6 (H1032X/A1049X), said variant confers herbicide resistance to a Cannabis plant.

43. Use of a HR-related gene comprising a nucleotide sequence selected from SEQ ID NO: 1, 4, 7 and 10 in the generation of a Cannabis herbicide-resistant plant, by base-editing the at least one HR-related gene so as to generate one or more nucleotide substitutions in the HR-related gene, said one or more nucleotide substitutions confer herbicide resistance to the Cannabis plant.

44. The use according to claim 43, wherein said use comprises the usage of a variant or mutant, an isolated nucleic acid sequence encoding the variant or mutant and/or an expression cassette and/or construct comprising a nucleotide sequence encoding the variant or mutant, in the generation of Cannabis herbicide-resistant plants, said variant or mutant is selected from the group comprising:
v. variant of SEQ ID NO:3 comprising amino acid mutation at one of more positions selected from A118X, P193X, A201X, W570X and or S649X, said variant confers herbicide resistance to a Cannabis plant;
vi. variant of SEQ ID NO:6 comprising amino acid mutation at one of more positions selected from S998X and S1052X, said variant confers herbicide resistance to a Cannabis plant;
vii. variant of SEQ ID NO:9 comprising amino acid mutation at one of more positions selected from T181X and P185X, said variant confers herbicide resistance to a Cannabis plant; and
viii. variant of SEQ ID NO:12 comprising amino acid mutation at one of more positions selected from SGR4 (K1033X/K1034X/G1035X), SGR5 (H1032X), and SGR6 (H1032X/A1049X), said variant confers herbicide resistance to a Cannabis plant.

45. A transgenic Cannabis plant or seed, comprising the genetically modified cell of claim 41.

46-70. (canceled)
